(19) United States
(12) Patent Application Publication
(10) Pub. No.: US 2025/0283089 A1
(43) Pub. Date: Sep. 11, 2025

(54) TREATMENT OF A NON-ALCOHOLIC FATTY LIVER DISEASE

(71) Applicant: ARROWHEAD PHARMACEUTICALS, INC., Pasadena, CA (US)

(72) Inventors: James C. HAMILTON, Arcadia, CA (US); Bruce D. GIVEN, Charleston, SC (US)

(21) Appl. No.: 18/854,700

(22) PCT Filed: Apr. 7, 2023
(86) PCT No.: PCT/US2023/065487

§ 371 Date: Oct. 7, 2024

Related U.S. Application Data
(60) Provisional application No. 63/329,081, filed on Apr. 8, 2022.
(60) Provisional application No. 63/401,858, filed on Aug. 29, 2022.

(51) Int. Cl. C12N 15/113; A61P 1/16

(52) U.S. Cl. CPC C12N 15/1137; A61P 1/16

(57) ABSTRACT

Described are methods of treatment of a non-alcoholic fatty liver disease via a reduction in HSD17B13 expression in a human subject in need of treatment.

### CROSS-REFERENCE TO RELATED APPLICATIONS

This application claims the benefit of U.S. Provisional Application No. 63/329,081 filed on Apr. 8, 2022, and U.S. Provisional Application No. 63/401,858 filed on Aug. 29, 2022, each of which is incorporated herein by reference in their entirety.

### SEQUENCE LISTING

This application contains a Sequence Listing which has been submitted in XML format and is hereby incorporated by reference in its entirety. The XML copy is named 70162W001_SL.xml, is about 115,715 bytes in size and was created on Mar. 7, 2023.

### FIELD OF THE INVENTION

Disclosed herein are methods of treatment of a non-alcoholic fatty liver disease (NAFLD), which can reduce (e.g., inhibit or silence) hepatic lipid droplet protein 17p-hydroxysteroid dehydrogenase type 13 (also referred to as HSD17B13, 17β-HSD13, HSD17β13, 17beta-HSD13, 17beta-HSD type 13, or 17B-HSD13) gene expression and its protein expression.

### BRIEF SUMMARY

In some aspects, the present disclosure provides a method for treating a non-alcoholic fatty liver disease in a human subject, comprising administering to the human subject a compound or a pharmaceutically acceptable salt thereof, at a dose of about 25 to about 200 mg, calculated based on a free acid form of the compound, wherein the compound comprises a double-stranded oligonucleotide linked to a ligand, wherein the double-stranded oligonucleotide reduces expression of HSD17β13 in the human subject, and wherein the ligand comprises a chemical structure of:

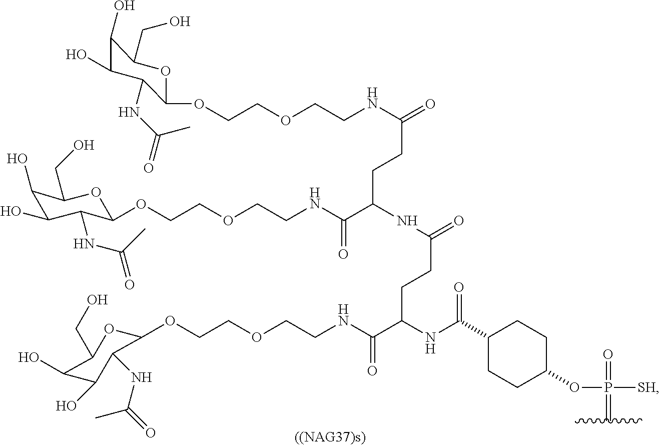

stereoisomer thereof, or a pharmaceutically acceptable salt thereof.

### BRIEF DESCRIPTION OF THE DRAWINGS

FIG. 1 shows effects of the test compound on expression of HSD17β13 mRNA at Day 71 relative to baseline in patients with NASH (non-alcoholic steatohepatitis) or NAFLD. The box displays Q1, median, and Q3. The cross mark displays the mean value, and the whiskers extend to the minimum and maximum values.

FIG. 2A and FIG. 2B show effects of the test compound on liver enzymes in patients with NASH or NAFLD on ALT (alanine aminotransferase) and AST (aspartate aminotransferase) respectively.

FIG. 3A and FIG. 3B show effects of the test compound on messenger RNA knockdown at Day 71 relative to baseline by genotype in patients with NASH or NAFLD, having HSD17β13 rs72613567 (FIG. 3A, the box displays Q1, median, and Q3, the cross mark displays the mean value, and the whiskers extend to the minimum and maximum values), and PNPLA3 rs738409 (FIG. 3B, the box displays Q1, median and Q3, the diamond displays the mean value, and the whiskers extend to the minimum and maximum values).

FIG. 4 shows the clinical study design and the dosing schedule of the test compound.

FIG. 5A to 5D shows a chemical structure of the test compound described in Example 2, in a free acid form. The circled numerical numbers indicate the connecting points of parts of the structure split into four pages due to length.

FIG. 6A to 6D shows a chemical structure of the test compound described in Example 2, in a sodium salt form. The circled numerical numbers indicate the connecting points of parts of the structure split into four pages due to length.

FIG. 7 shows a simulation of target engagement based on the clinical results in Example 2, to further support a dose of 200 mg every 12 weeks or 3 months.

### DETAILED DESCRIPTION

In some cases, treatment disclosed herein can reduce hepatocyte production of the HSD17β13 and provide a therapeutic and/or protective effect in human subjects having or suspected of NAFLD and potentially other chronic liver diseases. In some instances of human patients with NASH or suspected NASH (e.g., NAFLD with high ALT), the treatment can lead to reduction in HSD17β13 mRNA expression at about 25 to about 200 mg dose levels, based on postdose Day 71 biopsies, for example a near maximal reduction (e.g., at least about 90% reduction). For a dose disclosed herein for example 100 and 200 mg, the treatment inhibits HSD17β13 in subjects carrying the PNPLA3 rs738409 CG or GG genotypes that are associated with increased risk of NASH cirrhosis, and such inhibition effect can be substantially the same compared to subjects lacking PNPLA3 mutation. In some instances, reduced HSD17β13 expression can lead to or be accompanied by reductions in ALT and AST, which are surrogate markers of hepatic inflammation in NASH.

Unless indicated otherwise, the term “about” used herein means plus or minus 5% of a referred number.

Unless indicated otherwise, the term “a”, “an”, and “the” includes singular and plural references.

### Compounds for Use

Compounds used in treating human subjects disclosed herein can comprise, for example an RNA interference (RNAi) agent that reduces expression of HSD17B13 “HSD17B13 RNAi agent.” In some instances, the compound comprises HSD17β13-targeting double-stranded oligonucleotide linked to galactose-containing ligands that can be preferentially taken up by hepatocytes to promote liver specificity, following which the compound will engage the cell's RNAi machinery to target HSD17β13 mRNA for degradation, thereby decreasing the amount of free HSD17β13 mRNA for translation. These factors may minimize off-target effects or extrahepatic inhibition of the compound and contribute to the compound's acceptable safety profile. In some instances, the compound herein targets engagement with a knockdown of HSD17β13, mimicking a LOF (loss of function) mutation in NASH.

As used herein, the terms “reduce,” “silence,” “inhibit,” “down-regulate,” or “knockdown,” when referring to expression of HSD17B13, can mean that the expression of the gene, as measured by the level of mRNA (messenger RNA) transcribed from the gene, or the level of protein (or protein subunit) translated from the mRNA, in a cell, group of cells, tissue, organ, or subject in which the gene is transcribed, is reduced when the cell, group of cells, tissue, organ, or subject is treated with the RNAi agent as compared to a second cell, group of cells, tissue, organ, or subject that has not been so treated. HSD17B13 reduction may be measured by any appropriate assay or method known in the art. The non-limiting exemplary assays include microarray, Affymetrix gene chips, and qRT-PCR (quantitative Reverse Transcriptase Polymerase Chain Reaction) for HSD17B13 gene quantitation, and gel electrophoresis such as Western Blot and mass spectrometry for HSD17B13 protein measurements, as well as those assays set forth in International Patent Application Publication No. WO 2020/061177 (Patent Application No. PCT/US2019/051707), which is incorporated by reference herein in its entirety. A reference HSD17B13 mRNA gene transcript for normal humans can be transcript variant A, GenBank NM_178135.4. In some instances, the reduction in gene expression is measured by comparing the baseline levels of HSD17B13 mRNA or HSD17B13 protein in a human subject prior to a treatment disclosed herein, with the HSD17B13 mRNA or HSD17B13 protein levels after administration of the compound.

Compounds disclosed herein may contain one or more asymmetric centers (also referred to as a chiral centers) and may, therefore, exist as individual enantiomers, diastereoisomers, or other forms of stereoisomers, or as mixtures thereof. Chiral centers, such as chiral carbon atoms, may also be present in a substituent such as an alkyl group. Where the stereochemistry of a chiral center in any chemical structure illustrated herein, is not specified, the structure is intended to encompass any stereoisomer and all mixtures thereof. In some instances, compounds disclosed herein containing one or more chiral centers may be used as racemic modifications including racemic mixtures and racemates, enantiomerically-enriched mixtures, or as enantiomerically-pure individual stereoisomers. In some instances, a compound herein is provided as a salt, mixed salt, or free acid.

Individual stereoisomers which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. It will be appreciated that where the desired stereoisomer is converted into another chemical entity by one of the separation procedures described above, a further step is required to liberate the desired form. Alternatively, specific stereoisomers may be synthesized by asymmetric synthesis using optically active reagents, substrates, catalysts or solvents, or by converting one enantiomer to the other by asymmetric transformation. In some cases, a compound for use herein comprises a sense strand (also referred to as a passenger strand) and an antisense strand (also referred to as a guide strand) that are annealed to form a duplex.

In some instances, the compound comprises a double-stranded oligonucleotide, for example comprising an RNA, chemically modified RNA, DNA, or chemically modified DNA oligonucleotide molecule, any of which can have two strands of complementary sequence. In some instances, the double-stranded oligonucleotide is capable of degrading or inhibiting translation of messenger RNA (mRNA) transcripts of HSD17B13 mRNA in a sequence specific manner. In some instances, the compound disclosed herein may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that the HSD17B13 RNAi agents, as that term is used herein, operate primarily through the RNA interference mechanism, the disclosed RNAi agents are not bound by or limited to any particular pathway or mechanism of action. In some instances, the compound comprises a short or small interfering RNA (siRNA), double-strand RNA (dsRNA), micro RNA (miRNA), short hairpin RNA (shRNA), or dicer substrate. In some instances, the compounds for use herein include an antisense strand that has a region of complementarity to at least a portion of an HSD17B13 mRNA.

In some instances, a compound for use herein has a sense strand of 15 to 49 nucleotides in length, and an antisense strand of 18 to 49 nucleotides in length. In some instances, the sense and antisense strands are independently 18 to 26 nucleotides in length. In some instances, the sense and antisense strands are independently 21 to 26 nucleotides in length. In some instances, the sense and antisense strands are independently 21 to 24 nucleotides in length. In some instances, the sense strands are 15, 16, 17, 18, 19, 20, 21, 22, 23, 24, 25, 26, 27, 28, 29, or 30 nucleotides in length. In some instances, the antisense strands are 18, 19, 20, 21, 22, 23, 24, 25, 26, 27, 28, 29, or 30 nucleotides in length. In some instances, the sense strand and the antisense strand are both 21 nucleotides in length. The sense and antisense strands can be either the same length or different lengths. The sense and antisense strands can also form overhanging nucleotides on one or both ends of the nucleotide chain.

Compounds suitable for use herein can be covalently linked to a ligand that includes one or more N-acetyl-galactosamine moieties. The N-acetyl-galactosamine moieties can facilitate the targeting of the HSD17B13 RNAi agent to the asialoglycoprotein receptors (ASGPR) readily present on the surface of hepatocytes, which leads to internalization of the HSD17B13 RNAi agent by endocytosis or other means.

### Modified Nucleotides and Modified Internucleoside Linkages

Compounds for example HSD17B13 RNAi agents disclosed herein can comprise modified nucleotides, which can preserve activity of the RNAi agent while at the same time increasing the serum stability, as well as minimize the possibility of activating interferon activity in humans. As used herein, a “modified nucleotide” is a nucleotide other than a ribonucleotide (2′-hydroxyl nucleotide). In some instances, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include but are not limited to, deoxyribonucleotides, nucleotide mimics, 2′-modified nucleotides, inverted nucleotides, modified nucleobase-comprising nucleotides, bridged nucleotides, peptide nucleic acids (PNAs), 2′,3′seco nucleotide mimics (unlocked nucleobase analogues), locked nucleotides, 3′-O-methoxy (2′ internucleoside linked) nucleotides, 2′-F-arabino nucleotides, 5′-Me, 2′-fluoro nucleotides, morpholino nucleotides, vinyl phosphonate-containing nucleotides, and cyclopropyl phosphonate-containing nucleotides. In some instances, the modified nucleotides of an HSD17B13 RNAi agent are 2′-modified nucleotides (i.e., a nucleotide with a group other than a hydroxyl group at the 2′ position of the five-membered sugar ring). 2′-modified nucleotides include, but are not limited to, 2′Omethyl nucleotides, 2′-deoxy-2′-fluoro nucleotides (commonly referred to simply as 2′-Fluoro nucleotides), 2′-deoxy nucleotides, 2′-methoxyethyl (2′-O-2-methoxyethyl) nucleotides, 2′-amino nucleotides, and 2′-alkyl nucleotides. It may not be necessary for all nucleotides in a given RNAi agent to be uniformly modified. In some instances, more than one modification can be incorporated in a single HSD17B13 RNAi agent or even in a single nucleotide thereof. The HSD17B13 RNAi agent sense strands and antisense strands can be synthesized and/or modified by methods known in the art. Modification at one nucleotide can be independent of modification at another nucleotide.

In some instances, a nucleobase (often referred to as simply the “base”) disclosed herein may be modified. Natural nucleobases can include the primary purine bases adenine and guanine, and the primary pyrimidine bases cytosine, thymine, and uracil. A nucleobase may be modified to include, without limitation, universal bases, hydrophobic bases, promiscuous bases, size-expanded bases, and fluorinated bases. The synthesis of such modified nucleobases (including phosphoramidite compounds that include modified nucleobases) is known in the art.

Modified nucleobases include, for example, 5-substituted pyrimidines, 6-azapyrimidines and N-2, N-6 and 0-6 substituted purines, (e.g., 2aminopropyladenine, 5-propynyluracil, or 5-propynylcytosine), 5-methylcytosine (5-me-C), 5hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5propynyl uracil, 5-propynyl cytosine, 6-azo uracil, 6-azo cytosine, 6-azo thymine, 5-uracil (pseudouracil), 4-thiouracil, 8-halo, 8amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7methylguanine and 7-methyladenine, 8-azaguanine and 8-azaadenine, 7deazaguanine, 7deazaadenine, 3-deazaguanine, and 3-deazaadenine.

In some instances, all or substantially all the nucleotides of a compound disclosed herein for example an HSD17B13 RNAi agent are modified nucleotides. As used herein, an RNAi agent wherein substantially all the nucleotides are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all the nucleotides are modified nucleotides is a sense strand having two or fewer (i.e., 0, 1, or 2) nucleotides in the sense strand being ribonucleotides. As used herein, an antisense sense strand wherein substantially all the nucleotides are modified nucleotides is an antisense strand having two or fewer (i.e., 0, 1, or 2) nucleotides in the sense strand being ribonucleotides.

In some instances, one or more nucleotides of a compound disclosed herein for example an HSD17B13 RNAi agent are linked by non-standard linkages or backbones (i.e., modified internucleoside linkages or modified backbones). Modified internucleoside linkages or backbones include, but are not limited to, phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some instances, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some instances, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some instances, a sense strand of a compound disclosed herein for example an HSD17B13 RNAi agent can contain 1, 2, 3, 4, 5, or 6 phosphorothioate linkages, an antisense strand of an HSD17B13 RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some instances, a sense strand of an HSD17B13 RNAi agent can contain 1, 2, 3, or 4 phosphorothioate linkages, an antisense strand of an HSD17B13 RNAi agent can contain 1, 2, 3, or 4 phosphorothioate linkages, or both the sense strand and the antisense strand independently can contain 1, 2, 3, or 4 phosphorothioate linkages.

In some instances, a sense strand in a compound disclosed herein for example an HSD17B13 RNAi agent contains at least two phosphorothioate internucleoside linkages. In some instances, the at least two phosphorothioate internucleoside linkages are between the nucleotides at positions 1-3 from the 3′ end of the sense strand. In some instances, the at least two phosphorothioate internucleoside linkages are between the nucleotides at positions 1-3, 2-4, 3-5, 4-6, 4-5, or 6-8 from the 5′ end of the sense strand. In some instances, phosphorothioate internucleoside linkages are used to link the terminal nucleotides in the sense strand to capping residues present at the 5′-end, the 3′-end, or both the 5′- and 3′-ends of the nucleotide sequence. In some instances, phosphorothioate internucleoside linkages are used to link a ligand to the sense strand.

In some instances, an antisense strand of a compound disclosed herein for example an HSD17B13 RNAi agent contains three or four phosphorothioate internucleoside linkages. In some instances, the antisense strand contains three phosphorothioate internucleoside linkages. In some instances, the three phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some instances, the compound contains at least two phosphorothioate internucleoside linkages in the sense strand and three or four phosphorothioate internucleoside linkages in the antisense strand.

In some instances, a compound disclosed herein for example an HSD17B13 RNAi agent contains one or more modified nucleotides and one or more modified internucleoside linkages. In some instances, a 2′modified nucleoside is combined with modified internucleoside linkage.

TABLE 1A
Exemplary Antisense Strand Sequence

| SEQ | Antisense Sequence | SEQ | Underlying |
|---|---|---|---|
| ID | (Modified) | ID | Base Sequence |
| NO. | (5′→3′) | NO. | (5′→3′) |
| 1 | usCfsasUfcUfaucagA | 2 | UCAUCUAUCAGAC |
| | fcUfuCfuUfaCfsg | | UUCUUACG |

TABLE 1B
Exemplary Sense Strand Nucleotide Sequence
(shown as modified version without inverted
abasic residues or NAG ligand)

| SEQ | Sense Sequence | SEQ | Underlying |
|---|---|---|---|
| ID | (Modified) | ID | Base Sequence |
| NO. | (5′→3′) | NO. | (5′→3′) |
| 3 | cguaagaaGfuCfuG | 4 | CGUAAGAAGUCUGA |
| | fauagauga | | UAGAUGA |

As used in Tables 1A, 1B, and 2 herein, the following notations are used to indicate modified nucleotides, ligand groups, and linking groups: A, C, G, and U represent adenosine, cytidine, guanosine, and uridine, respectively; a, c, g, and u represent 2′-O-methyl adenosine, cytidine, guanosine, and uridine, respectively; Af, Cf, Gf, and Uf represent 2′-fluoro adenosine, cytidine, guanosine, and uridine, respectively; s represents a phosphorothioate linkage; (invAb) represents an inverted abasic deoxyribose residue (see Table 2); and (NAG37)s represents the structure shown in Table 3, below.

Unless otherwise indicated by the sequence (such as, for example, by a phosphorothioate linkage “s”), when present in a strand, the monomers can be mutually linked by 5′-3′-phosphodiester bonds. In some instances, the inclusion of a phosphorothioate linkage as shown in the modified nucleotide sequences disclosed herein replaces the phosphodiester linkage typically present in oligonucleotides. In some instances, the terminal nucleotide at the 3′ end of a given oligonucleotide sequence would typically have a hydroxyl (—OH) group at the respective 3′ position of the given monomer instead of a phosphate moiety ex vivo. In some instances, when viewing the respective strand 5′→3′, the inverted abasic residues are inserted such that the 3′ position of the deoxyribose is linked at the 3′ end of the preceding monomer on the respective strand. Moreover, as the person of ordinary skill would readily understand and appreciate, while the phosphorothioate chemical structures depicted herein typically show the anion on the sulfur atom, the present disclosure encompasses phosphorothioate tautomers (e.g., where the sulfur atom has a double-bond and the anion is on an oxygen atom). In some instances, each sense strand and/or antisense strand can have any linking groups disclosed herein, linked to the 5′ and/or 3′ end of the sequence.

### Capping Residues

In some instances, a sense strand disclosed herein may include one or more capping residues, sometimes referred to as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent with certain beneficial properties, for example, protection against exonuclease degradation. In some instances, inverted abasic residues (invAb) (also referred to as “inverted abasic sites”) are added as capping residues (see Table 2). Capping residues include, for example, inverted abasic residues as well as carbon chains such as a terminal C₃H₇ (propyl), C₆H₁₃ (hexyl), or C₁₂H₂₅ (dodecyl) groups. In some instances, a capping residue is present at either the 5′ terminal end, the 3′ terminal end, or both the 5′ and 3′ terminal ends of the sense strand. In some instances, the 5′ end and/or the 3′ end of the sense strand may include more than one inverted abasic deoxyribose moiety as a capping residue.

In some instances, one or more inverted abasic residues (invAb) are added to the 3′ end of a sense strand. In some instances, one or more inverted abasic residues (invAb) are added to the 5′ end of the sense strand. In some instances, one or more inverted abasic residues or inverted abasic sites are inserted between the ligand and the nucleotide sequence of the sense strand of the RNAi agent. In some instances, the inclusion of one or more inverted abasic residues or inverted abasic sites at or near the terminal end or terminal ends of the sense strand of an RNAi agent allows for enhanced activity or other desired properties of an RNAi agent.

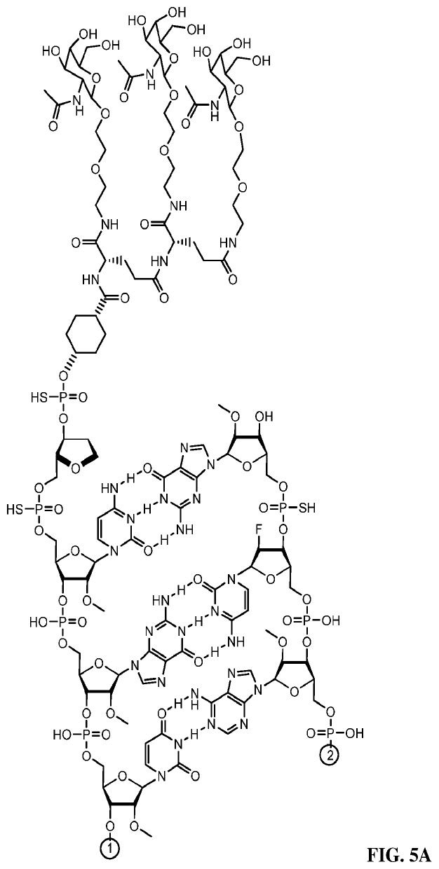

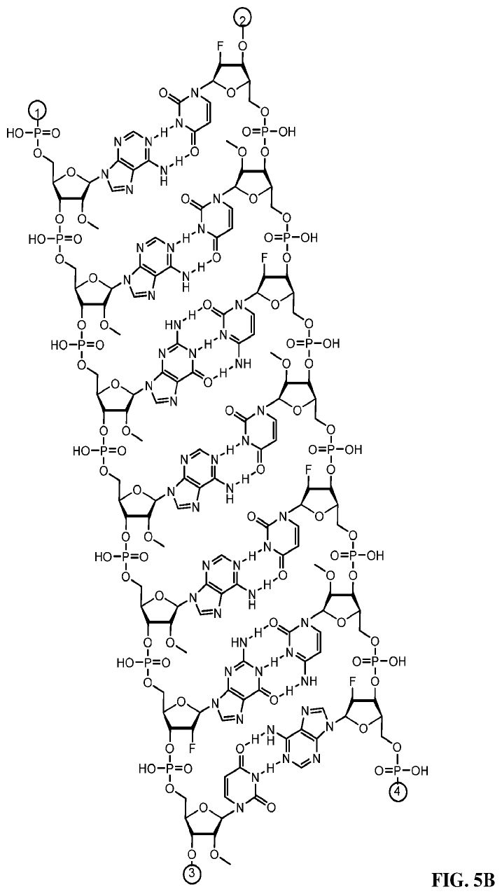

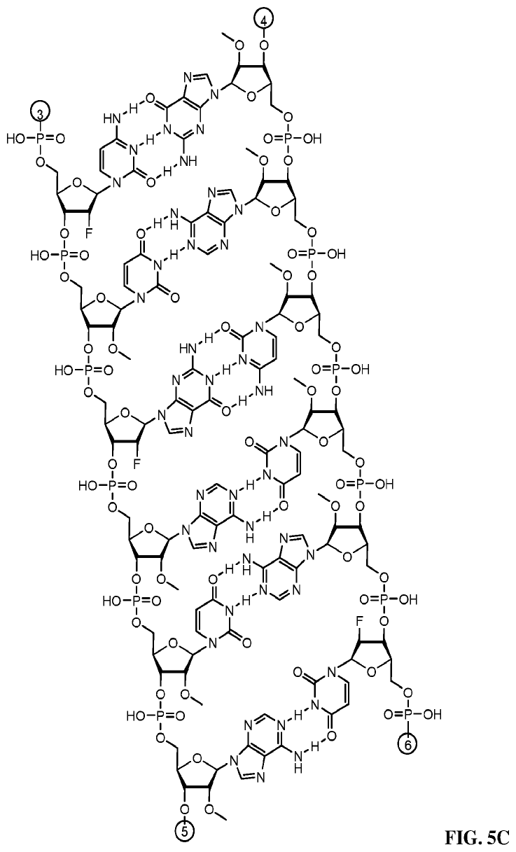

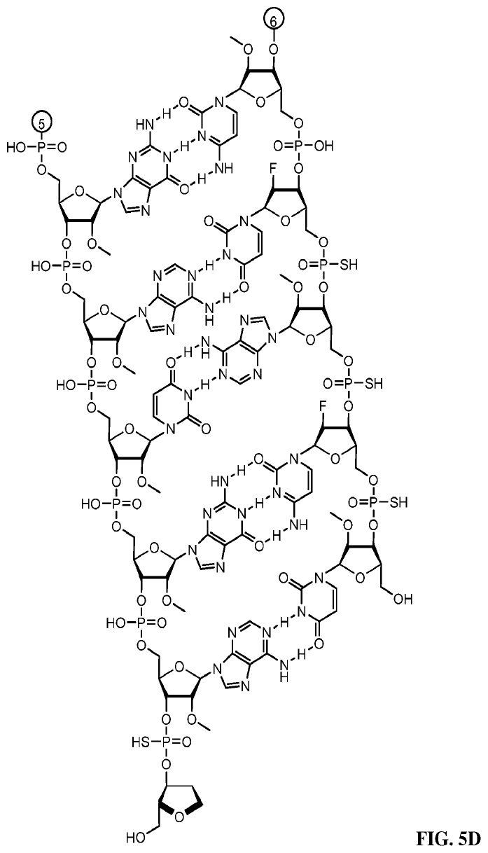

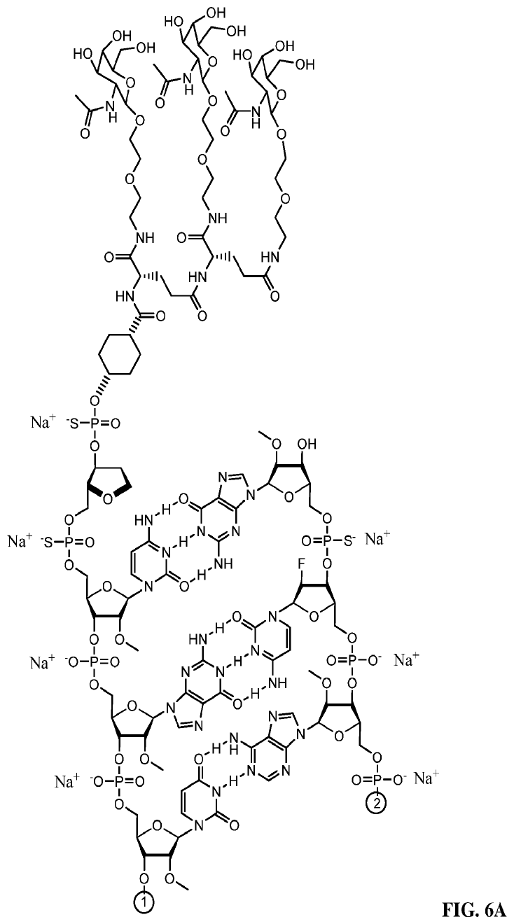

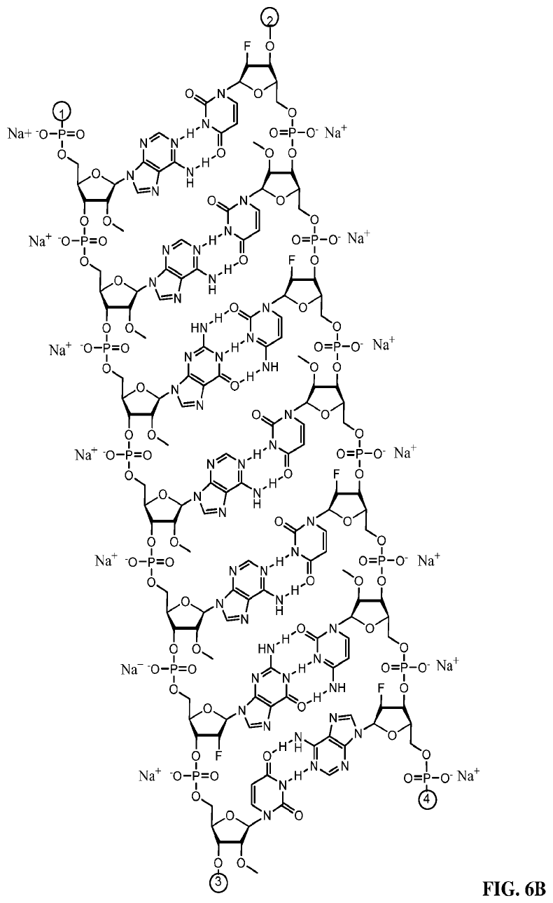

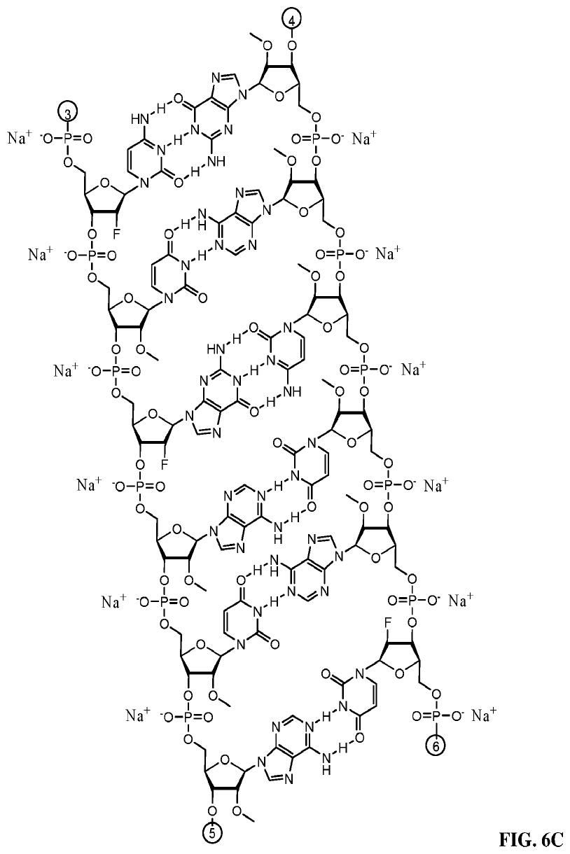

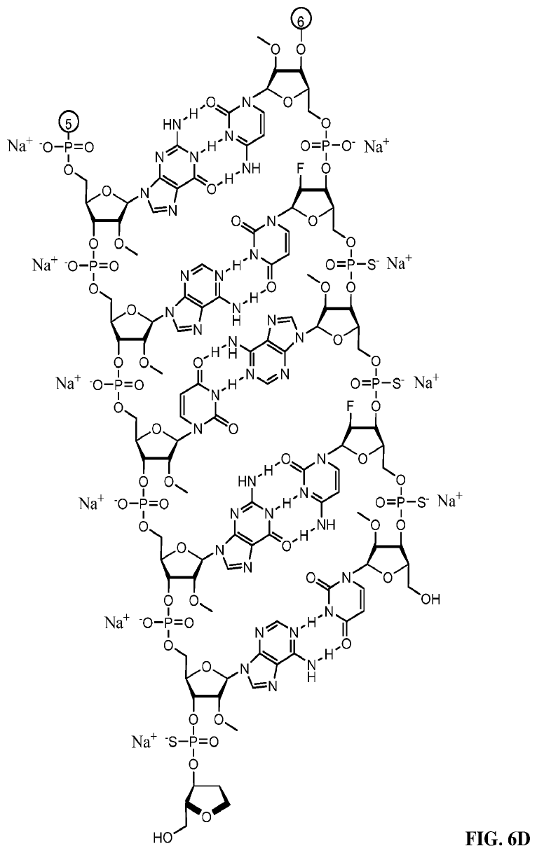

In some instances, one or more inverted abasic residues (invAb) are added to the 5′ end of a sense strand. In some instances, one or more inverted abasic residues can be inserted between the ligand and the nucleotide sequence of the sense strand of the RNAi agent. The inverted abasic residues may be linked via phosphate, phosphorothioate (e.g., shown herein as (invAb)s)), or other internucleoside linkages. The chemical structures for inverted abasic deoxyribose residues are shown in Table 2 below, as well as in the chemical structures shown in FIG. 5A to 5D and FIG. 6A to 6D.

TABLE 2
Inverted Abasic (Deoxyribose) Chemical Structures
When positioned internally:

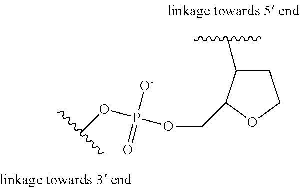 [SMILES: *OP(=O)([O-])OCC1OCCC1*]

(invAb)
When positioned internally:

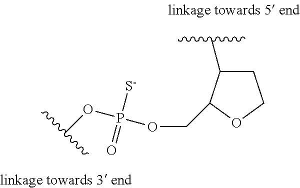 [SMILES: *OP(=O)([S-])OCC1OCCC1*]

(invAb)s
When positioned at the 3′ terminal end:

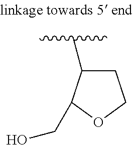 [SMILES: *C1CCOC1CO]

(invAb)

### Linked Ligands

In some cases, a compound for use herein can include a RNA molecule such as an HSD17B13 RNAi agent, linked to one or more non-nucleotide groups such as a ligand that can enhance targeting or delivery of the RNAi agent. Examples include three N-acetyl-galactosamine moieties, are described in the present disclosure. The ligand can be covalently linked to the 3′ and/or 5′ end of either the sense strand and/or the antisense strand. In some instances, the compound contains a ligand linked to the 3′ and/or 5′ end of the sense strand. In some instances, a ligand is linked to the 5′ end of the sense strand. In some instances, the ligand comprises, consists essentially of, or consists of the structure (NAG37)s, and is linked to the 5′ end of the sense strand. A ligand can be linked directly or indirectly to the RNAi agent via a linker/linking group. In some instances, a ligand is linked to the RNAi agent via a labile, cleavable, or reversible bond or linker. In some instances, a ligand is linked to an inverted abasic residue at the 5′ end of the sense strand.

Ligands disclosed herein can enhance the pharmacokinetic or biodistribution properties of a RNAi agent to which they are attached to improve cell-specific distribution and cell-specific uptake of the RNAi agent. In some instances, the ligand enhances endocytosis of the RNAi agent. In some instances, the ligand can be monovalent, divalent, trivalent, tetravalent, or have higher valency for the target to which it is directed. Representative ligands include, without limitation, compounds with affinity to cell surface molecules, cell receptor ligands, haptens, antibodies, monoclonal antibodies, antibody fragments, and antibody mimics with affinity to cell surface molecules.

The preparation of ligands, such as galactose derivative clusters that include N-acetyl-galactosamine, is described in, for example, International Patent Application Publication No. WO 2018/044350 (Patent Application No. PCT/US2017/021147) and International Patent Application Publication No. WO 2017/156012 (Patent Application No. PCT/US2017/021175), the contents of both of which are incorporated by reference herein in their entirety.

For example, a ligand linked to the HSD17B13 RNAi agent described in Tables 1A and 1B can have the chemical structure of (NAG37)s, as shown in Table 3.

TABLE 3
Chemical Structure of (NAG37)s.

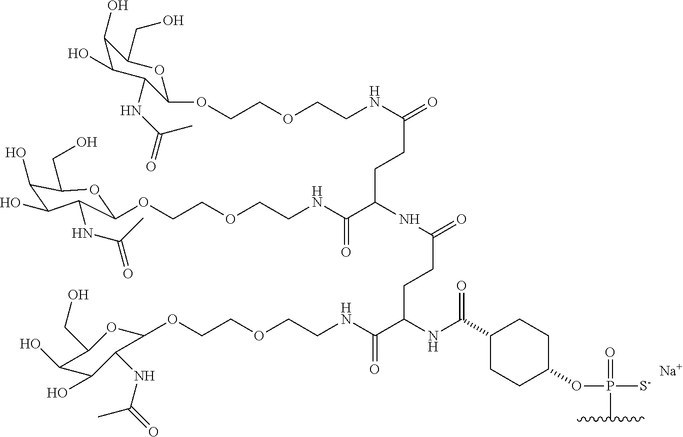

((NAG37)s shown in sodium salt form)

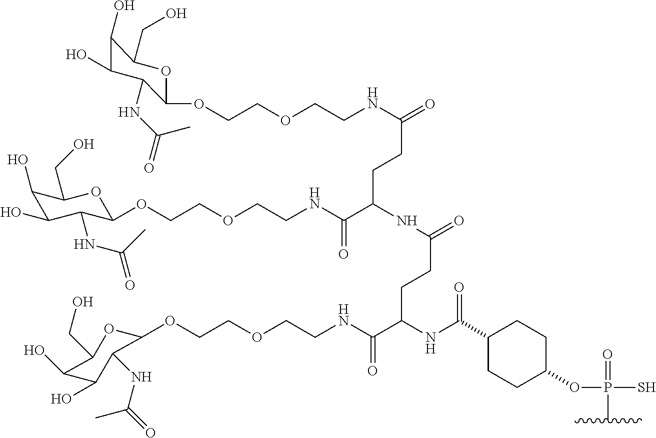

((NAG37)s shown in free acid form)

### Pharmaceutical Compositions

The compounds for example HSD17B13 RNAi agents suitable for use herein can be formulated as pharmaceutical compositions for administration to human subjects. The pharmaceutical compositions can be used to treat or prevent a subject having a disease or disorder that would benefit from inhibition of expression of HSD17B13 mRNA or reduction in the level of HSD17B13 protein, such as human subjects having a non-alcoholic fatty liver disease. In some instances, one or more pharmaceutically acceptable excipients (including vehicles, carriers, diluents, and/or delivery polymers) are added to the pharmaceutical compositions that include an HSD17B13 RNAi agent, thereby forming a pharmaceutical formulation suitable for in vivo delivery to a human subject.

As used herein, “pharmacologically effective amount,” “therapeutically effective amount,” or simply “effective amount” refers to that amount of an active agent to produce a pharmacological, therapeutic or preventive result. In some instances, a therapeutically or prophylactically effective amount of one or more of pharmaceutical compositions is administered to a subject in need thereof, to decrease the number, severity, and/or frequency of symptoms of a disease in the subject.

As used herein, a pharmaceutical composition or medicament includes a pharmacologically effective amount of at least one compound disclosed herein and one or more pharmaceutically acceptable excipients. Excipients are substances other than the Active Pharmaceutical Ingredient (API, therapeutic product, e.g., HSD17B13 RNAi agent) that are intentionally included in the drug delivery system. Excipients do not exert or are not intended to exert a therapeutic effect at the intended dosage. Excipients can act to a) aid in processing of the drug delivery system during manufacture, b) protect, support, or enhance stability, bioavailability or patient acceptability of the API, c) assist in product identification, and/or d) enhance any other attribute of the overall safety, effectiveness, of delivery of the API during storage or use.

Excipients may include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

Pharmaceutical compositions suitable for injection include sterile aqueous solutions (where water soluble). For subcutaneous or intravenous or intramuscular administration, suitable carriers may include physiological saline, bacteriostatic water, Cremophor® EL™ (BASF, Parsippany, NJ) or phosphate buffer. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof.

Sterile injectable solutions can be prepared by incorporating the active compound in the required amount in an appropriate solvent with one or a combination of ingredients enumerated above, as required, followed by filter sterilization. In some instances, dispersions are prepared by incorporating the active compound into a sterile vehicle, which contains a basic dispersion medium and the required other ingredients from those enumerated above.

A pharmaceutical composition can contain other additional components commonly found in pharmaceutical compositions. Such additional components be selected from active agents such as anti-purities, astringents, local anesthetics, anti-inflammatory agents, or antihistamines.

The described pharmaceutically acceptable formulations can be packaged into kits, containers, packs, or dispensers. The pharmaceutical compositions described herein can be packaged in pre-filled syringes or vials.

### Methods of Treatment

As used herein, the terms “treat,” “treatment,” “treating,” and the like, may include the prevention, management, prophylactic treatment, reduction, and/or inhibition of the number, severity, and/or frequency of a disease and/or one or more symptoms of a disease in a subject. In some cases, the terms “treat,” “treatment,” “treating,” and the like mean management, reduction, and/or inhibition of the number, severity, and/or frequency of a disease and/or one or more symptoms of a disease in a subject. Unless otherwise expressly noted, treatment also can include relief from or alleviation of underlying conditions and/or symptoms of the disease.

Embodiment 1: A method for treating a non-alcoholic fatty liver disease in a human subject in need thereof, comprising administering to the human subject a compound or a pharmaceutically acceptable salt thereof, at a dose of about 25 to about 200 mg, wherein the dose is calculated based on a free acid form of the compound, wherein the compound comprises a double-stranded oligonucleotide linked to a ligand, wherein the double-stranded oligonucleotide reduces expression of HSD17β13 in the human subject, and wherein the ligand comprises a chemical structure of:

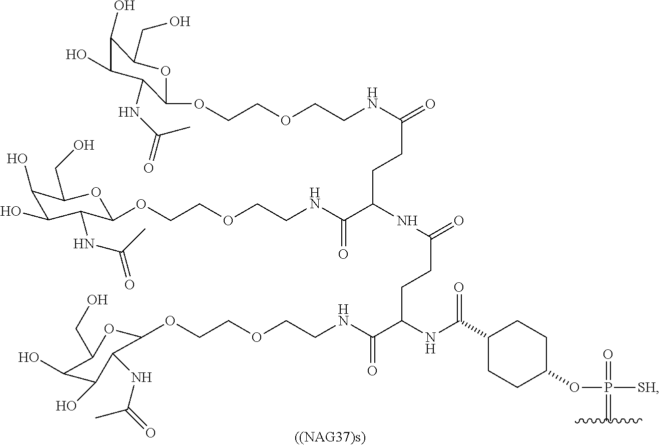

stereoisomer thereof, or a salt (e.g., pharmaceutically acceptable salt) thereof.

Embodiment 2: The method of Embodiment 1, wherein the administration is a single dose.

Embodiment 3: The method of Embodiment 1, wherein the administration is repeated once, e.g., at an interval of about 28 days (i.e., 4 weeks), about 12 weeks, or about 3 months, between doses, for example about 100 mg every 4 weeks, or about 200 mg every 12 weeks, optionally for about 52 weeks or about 1 year. In some instances, the administration is repeated every 4 weeks, e.g., at the dose of 100 mg. In some instances, the administration is repeated every 12 weeks, e.g., at the dose of 200 mg.

Embodiment 4: The method of any one of Embodiments 1-3, wherein the dose is about 50 mg to about 100 mg, for example about 50 mg.

Embodiment 5: The method of any one of Embodiments 1-3, wherein the dose is about 100 mg to about 200 mg, for example about 100 mg.

Embodiment 6: The method of any one of Embodiments 1-3, wherein the dose is about 50 mg to about 200 mg, for example about 200 mg.

Embodiment 7: The method of any one of Embodiments 1-6, wherein the compound is in a sodium salt form.

Embodiment 8: The method of any one of Embodiments 1-7, wherein the compound is administered by an injection, for example at a concentration of 200 mg/ml.

Embodiment 9: The method of Embodiment 8, wherein the injection is a subcutaneous injection.

Embodiment 10: The method of any one of Embodiments 1-9, wherein the compound is present in a unit dosage form, for example including a single (unit) dosage form.

Embodiment 11: The method of any one of Embodiments 1-10, wherein the non-alcoholic fatty liver disease is non-alcoholic steatohepatitis (NASH), e.g., pre-cirrhotic non-alcoholic steatohepatitis, NASH-F3, NASH with bridging fibrosis, or biopsy-proven NASH.

Embodiment 12: The method of any one of Embodiments 1-11, wherein the human subject has a HSD17β13 rs72613567 mutation, PNPLA3 rs738409 (I148M) mutation, or a combination thereof. In some instances, the human subject has a HSD17β13 rs72613567 mutation. In some instances, the human subject has a PNPLA3 rs738409 (I148M) mutation.

Embodiment 13: The method of any one of Embodiments 1-12, wherein the double-stranded oligonucleotide comprises an antisense strand that comprises a modified nucleotide sequence that differs by 0 or 1 nucleotides from one of the following sequences (5′→3′):

(nucleotide sequence SEQ ID NO: 1)
usCfsasUfcUfaucagAfcUfuCfuUfaCfsg;
(nucleotide sequence SEQ ID NO: 5)
usCfsasUfcUfaUfcAfgAfcUfuCfuUfaCfsg;
(nucleotide sequence SEQ ID NO: 6)
usGfsasUfcCfaAfaAfaUfgUfcCfuAfgGfsc;
or
(nucleotide sequence SEQ ID NO: 7)
usGfsasUfcCfaaaaaUfgUfcCfuAfgGfsc;

and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

Embodiment 14: The method of any one of Embodiments 1-13, wherein the double-stranded oligonucleotide comprises a sense strand that comprises a modified nucleotide sequence that differs by 0 or 1 nucleotides from one of the following sequences (5′→3′):

(nucleotide sequence SEQ ID NO: 3)
cguaagaaGfuCfuGfauagauga;
(nucleotide sequence SEQ ID NO: 8)
cguaagaaGfUfCfugauagauga;
(nucleotide sequence SEQ ID NO: 9)
gccuaggaCfAfUfuuuugiauca;
or
(nucleotide sequence SEQ ID NO: 10)
gccuaggaCfaUfuUfuugiauca;

and wherein a, c, g, i, and u represent 2′-O-methyl adenosine, 2′-O-methyl cytidine, 2′-O-methyl guanosine, 2′-O-methyl inosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

Embodiment 15: The method of any one of Embodiments 1-14, wherein the double-stranded oligonucleotide comprises an antisense strand that comprises a sequence: usCfsasUfcUfaucagAfcUfuCfuUfaCfsg (nucleotide sequence SEQ ID NO:1), and a sense strand that comprises a sequence: (NAG37)s(invAb)scguaagaaGfuCfuGfauagaugas(invAb) (nucleotide sequence SEQ ID NO:11),
and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; s represents a phosphorothioate linkage; and (invAb) represents an inverted abasic deoxyribose residue.

Embodiment 16: The method of any one of Embodiments 1-15, wherein the method reduces the expression level of HSD17β13 mRNA in the human subject by at least about 50% (e.g., at least about 62%, at least about 79%, at least about 84%, at least about 90%, or at least about 96%) compared with that prior to the administration, as measured by quantitative Reverse Transcriptase Polymerase Chain Reaction (qRT-PCR) with a liver biopsy sample from the human subject, for example about day 71 or 113 from the first administration.

Embodiment 17: The method of any one of Embodiments 1-16, wherein the method reduces the expression of HSD17β13 protein in the human subject by at least about 30% (e.g., at least about 50%, at least about 60%, at least about 83%, at least about 92%, or at least about 97%) compared with that prior to the administration, as measured by Western Blot with a liver biopsy sample from the human subject, for example about day 71 or 113 from the first administration.

Embodiment 18: The method of any one of Embodiments 1-17, wherein the method reduces ALT (alanine aminotransferase) level in the human subject by at least about 7.7% (e.g., at least about 14%, at least about 26%, at least about 36%, or at least about 39%), or at least about 40% (e.g., at least about 51%, at least about 53%, or at least about 60%) compared with that prior to the administration, as measured in the human subject's serum, for example about day 71 or 113 from the first administration.

Embodiment 19: The method of anyone of Embodiments 1-18, wherein the method reduces AST (aspartate aminotransferase) level in the human subject by at least about 7.5% (e.g., at least about 15%), or at least about 20% (e.g., at least about 24%, or at least about 28%) compared with that prior to the administration, as measured in the human subject's serum, for example about day 71 or 113 from the first administration.

Embodiment 20: The method of any one of Embodiments 1-19, wherein the method reduces or slows an increase of the human subject's liver fat fraction (e.g., a reduction of at least 4-41% or at least 7-8%) compared with that prior to the administration, as measured by magnetic resonance imaging, for example about day 71 or 113 from the first administration.

Embodiment 21: The method of anyone of Embodiments 1-20, wherein the method reduces or slows an increase of the human subject's liver stiffness (e.g., a reduction of at least 4-37%) compared with that prior to the administration, as measured by transient elastography, for example about day 71 or 113 from the first administration.

Embodiment 22: The method of any one of Embodiments 1-21, wherein the chemical structure of the ligand comprises:

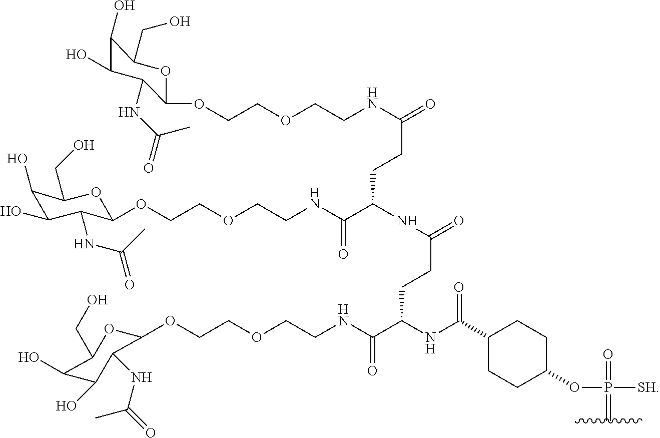

Embodiment 23: The method of Embodiment 22, wherein the compound is in a free acid form having a chemical structure as illustrated in FIG. 5A to 5D.

Embodiment 24: The method of any one of Embodiments 1-21, wherein the chemical structure of the ligand comprises:

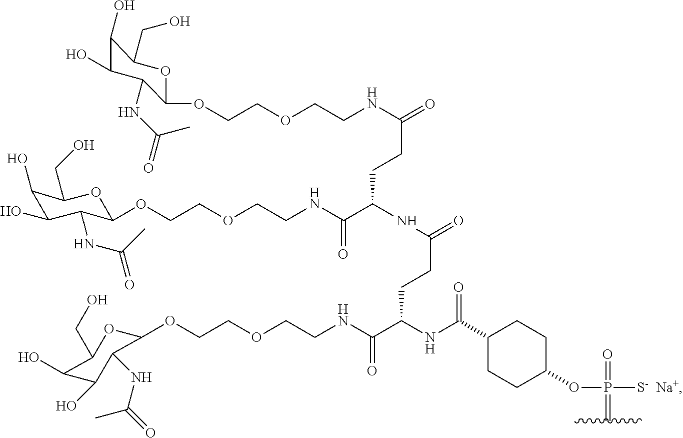

Embodiment 25: The method of Embodiment 24, wherein the compound is in a sodium salt form having a chemical structure as illustrated in FIG. 6A to 6D.

Embodiment 26: The method of any one of Embodiments 1-25, wherein the method reduces GGT (gamma-glutamyl transferase) level in the human subject by at least about 1.4% or at least 8.3% or at least 9%, compared with that prior to the administration, as measured in the human subject's serum, for example about day 71 or 113 from the first use.

Embodiment 27: A compound or a pharmaceutically acceptable salt thereof for use in treating non-alcoholic fatty liver disease in a human subject, wherein the use comprises administering to the human subject the compound or pharmaceutically acceptable salt thereof, at a dose of about 25 to about 200 mg, wherein the dose is calculated based on a free acid form of the compound, wherein the compound comprises a double-stranded oligonucleotide linked to a ligand, wherein the double-stranded oligonucleotide reduces expression of HSD17β13 in the human subject, and wherein the ligand comprises a chemical structure of:

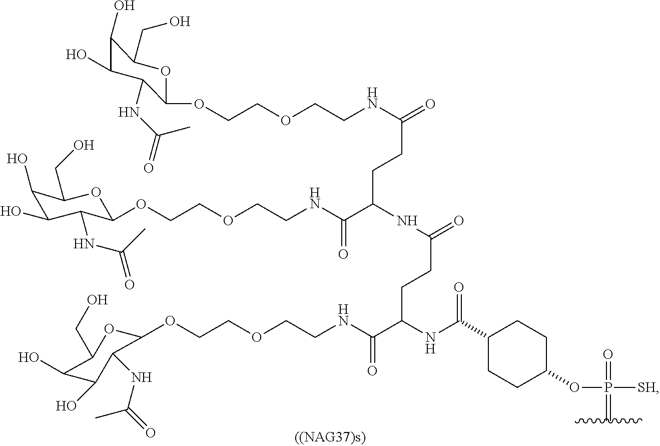

stereoisomer thereof, or a salt (e.g., a pharmaceutically acceptable salt) thereof.

Embodiment 28: The compound or a pharmaceutically acceptable salt thereof for use according to Embodiment 27, wherein the administration is a single dose.

Embodiment 29: The compound or a pharmaceutically acceptable salt thereof for use according to Embodiment 28, wherein the administration is repeated once, e.g., at an interval of about 28 days (i.e., 4 weeks), about 12 weeks, or about 3 months, between doses, for example about 100 mg every 4 weeks, or about 200 mg every 12 weeks, optionally for about 52 weeks or about 1 year. Embodiment 29a: The compound or a pharmaceutically acceptable salt thereof for use according to Embodiment 28, wherein the administration is about 100 mg every 4 weeks for about 52 weeks. Embodiment 29b: The compound or a pharmaceutically acceptable salt thereof for use according to Embodiment 28, wherein the administration is about 200 mg every 12 weeks for about 52 weeks.

Embodiment 30: The compound or a pharmaceutically acceptable salt thereof for use according to any one of Embodiments 27-29, wherein the dose is about 50 mg to about 100 mg, for example about 50 mg.

Embodiment 31: The compound or a pharmaceutically acceptable salt thereof for use according to any one of Embodiments 27-29, wherein the dose is about 100 mg to about 200 mg, for example about 100 mg.

Embodiment 32: The compound or a pharmaceutically acceptable salt thereof for use according to any one of Embodiments 27-29, wherein the dose is about 50 mg to about 200 mg, for example about 200 mg.

Embodiment 33: The compound or a pharmaceutically acceptable salt thereof for use according to any one of Embodiments 27-32, wherein the compound is in a sodium salt form.

Embodiment 34: The compound or a pharmaceutically acceptable salt thereof for use according to any one of Embodiments 27-33, wherein the compound is administered by an injection, for example at a concentration of 200 mg/ml.

Embodiment 35: The compound or a pharmaceutically acceptable salt thereof for use according to Embodiment 34, wherein the injection is subcutaneous.

Embodiment 36: The compound or a pharmaceutically acceptable salt thereof for use according to any one of Embodiments 27-35, wherein the compound is administered in a unit dosage form, e.g., present in a single unit dosage form.

Embodiment 37: The compound or a pharmaceutically acceptable salt thereof for use according to any one of Embodiments 27-36, wherein the non-alcoholic fatty liver disease is non-alcoholic steatohepatitis (NASH), e.g., pre-cirrhotic non-alcoholic steatohepatitis, NASH-F3, NASH with bridging fibrosis, or biopsy-proven NASH.

Embodiment 38: The compound or a pharmaceutically acceptable salt thereof for use according to any one of Embodiments 27-37, wherein the human subject has a HSD17β13 rs72613567 mutation, PNPLA3 rs738409 (I148M) mutation, or a combination thereof.

Embodiment 39: The compound or a pharmaceutically acceptable salt thereof for use according to any one of Embodiments 27-38, wherein the double-stranded oligonucleotide comprises an antisense strand that comprises a modified nucleotide sequence that differs by 0 or 1 nucleotides from one of the following sequences (5′→3′):

(nucleotide sequence SEQ ID NO: 1)
usCfsasUfcUfaucagAfcUfuCfuUfaCfsg;
(nucleotide sequence SEQ ID NO: 5)
usCfsasUfcUfaUfcAfgAfcUfuCfuUfaCfsg;
(nucleotide sequence SEQ ID NO: 6)
usGfsasUfcCfaAfaAfaUfgUfcCfuAfgGfsc;
or
(nucleotide sequence SEQ ID NO: 7)
usGfsasUfcCfaaaaaUfgUfcCfuAfgGfsc;

and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

Embodiment 40: The compound or a pharmaceutically acceptable salt thereof for use according to any one of Embodiments 27-39, wherein the double-stranded oligonucleotide comprises a sense strand that comprises a modified nucleotide sequence that differs by 0 or 1 nucleotides from one of the following sequences (5′→3′):

(nucleotide sequence SEQ ID NO: 3)
cguaagaaGfuCfuGfauagauga;
(nucleotide sequence SEQ ID NO: 8)
cguaagaaGfUfCfugauagauga;
(nucleotide sequence SEQ ID NO: 9)
gccuaggaCfAfUfuuuugiauca;
or
(nucleotide sequence SEQ ID NO: 10)
gccuaggaCfaUfuUfuugiauca;

and wherein a, c, g, i, and u represent 2′-O-methyl adenosine, 2′-O-methyl cytidine, 2′-O-methyl guanosine, 2′-O-methyl inosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

Embodiment 41: The compound or a pharmaceutically acceptable salt thereof for use according to any one of Embodiments 27-40, wherein the double-stranded oligonucleotide comprises an antisense strand that comprises a sequence: usCfsasUfcUfaucagAfcUfuCfuUfaCfsg (nucleotide sequence SEQ ID NO:1), and a sense strand that comprises a sequence: (NAG37)s(invAb)scguaagaaGfuCfuGfauagaugas(invAb) (nucleotide sequence SEQ ID NO:11),
and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; s represents a phosphorothioate linkage; and (invAb) represents an inverted abasic deoxyribose residue.

Embodiment 42: The compound or a pharmaceutically acceptable salt thereof for use according to any one of Embodiments 27-41, wherein the use reduces the expression of HSD17β13 mRNA in the human subject by at least about 50% (e.g., at least about 62%, at least about 79%, at least about 84%, at least about 90%, or at least about 96%) compared with that prior to the administration, as measured by quantitative Reverse Transcriptase Polymerase Chain Reaction (qRT-PCR) with a liver biopsy sample from the human subject, for example about day 71 or 113 from the first administration.

Embodiment 43: The compound or a pharmaceutically acceptable salt thereof for use according to any one of Embodiments 27-42, wherein the use reduces the expression of HSD17β13 protein in the human subject by at least about 30% (e.g., at least about 50%, at least about 60%, at least about 83%, at least about 92%, or at least about 97%) compared with that prior to the administration, as measured by Western Blot with a liver biopsy sample from the human subject, for example about day 71 or 113 from the first administration.

Embodiment 44: The compound or a pharmaceutically acceptable salt thereof for use according to any one of Embodiments 27-43, wherein the use reduces ALT (alanine aminotransferase) level in the human subject by at least about 7.7% (e.g., at least about 14%, at least about 26% at least about 36%, or at least about 39%), or at least about 40% (e.g., at least about 51%, at least about 53%, or at least about 60%) compared with that prior to the administration, as measured in the human subject's serum, for example about day 71 or 113 from the first administration.

Embodiment 45: The compound or a pharmaceutically acceptable salt thereof for use according to any one of Embodiments 27-44, wherein the use reduces AST (aspartate aminotransferase) level in the human subject by at least about 7.5% (e.g., at least about 15%), or at least about 20% (e.g., at least about 24%, or at least about 28%) compared with that prior to the administration, as measured in the human subject's serum, for example about day 71 or 113 from the first administration.

Embodiment 46: The compound or a pharmaceutically acceptable salt thereof for use according to any one of Embodiments 27-45, wherein the use reduces or slows an increase of the human subject's liver fat fraction (e.g., a reduction of at least 4-41% or at least about 7-8%) compared with that prior to the administration, as measured by magnetic resonance imaging, for example about day 71 or 113 from the first administration.

Embodiment 47: The compound or a pharmaceutically acceptable salt thereof for use according to any one of Embodiments 27-46, wherein the use reduces or slows an increase of the human subject's liver stiffness (e.g., a reduction of at least 4-37%) compared with that prior to the administration, as measured by transient elastography, for example about day 71 or 113 from the first administration.

Embodiment 48: The compound or a pharmaceutically acceptable salt thereof for use according to any one of Embodiments 27-47, wherein the chemical structure of the ligand comprises:

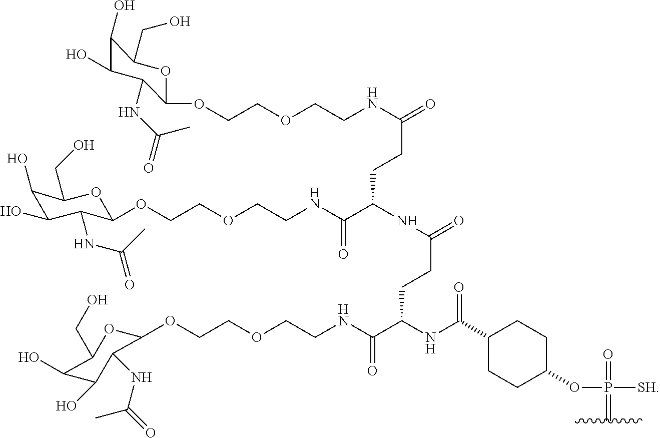

Embodiment 49: The compound or a pharmaceutically acceptable salt thereof for use according to Embodiment 48, wherein the compound is in a free acid form having a chemical structure as illustrated in FIG. 5A to 5D.

Embodiment 50: The compound or a pharmaceutically acceptable salt thereof for use according to any one of Embodiments 27-47, wherein the chemical structure of the ligand comprises:

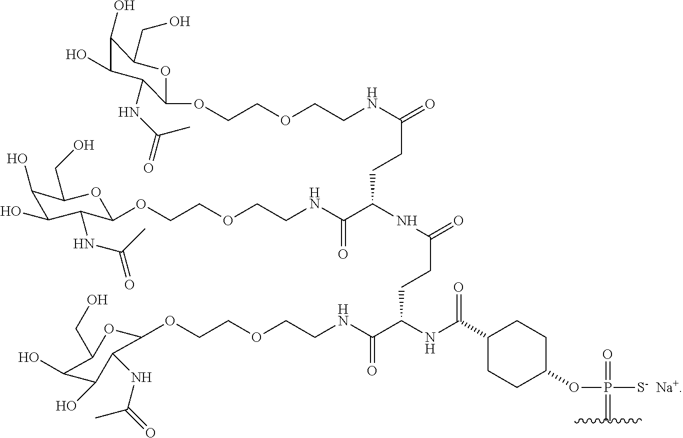

Embodiment 51: The compound or a pharmaceutically acceptable salt thereof for use according to Embodiment 50, wherein the compound is in a sodium salt form having a chemical structure as illustrated in FIG. 6A to 6D.

Embodiment 52: The compound or a pharmaceutically acceptable salt thereof for use according to any one of Embodiments 27-51, wherein the use reduces GGT (gamma-glutamyl transferase) level in the human subject by at least about 1.4% or at least 8.3% or at least 9%, compared with that prior to the administration, as measured in the human subject's serum, for example about day 71 or 113 from the first use.

Embodiment 53: Use of a compound or a pharmaceutically acceptable salt thereof for the manufacture of a medicament for the treatment of non-alcoholic fatty liver disease, wherein the compound or pharmaceutically acceptable salt thereof is used at a dose of about 25 to about 200 mg, wherein the dose is calculated based on a free acid form of the compound, wherein the compound comprises a double-stranded oligonucleotide linked to a ligand, wherein the double-stranded oligonucleotide reduces expression of HSD17β13, and wherein the ligand comprises a chemical structure of:

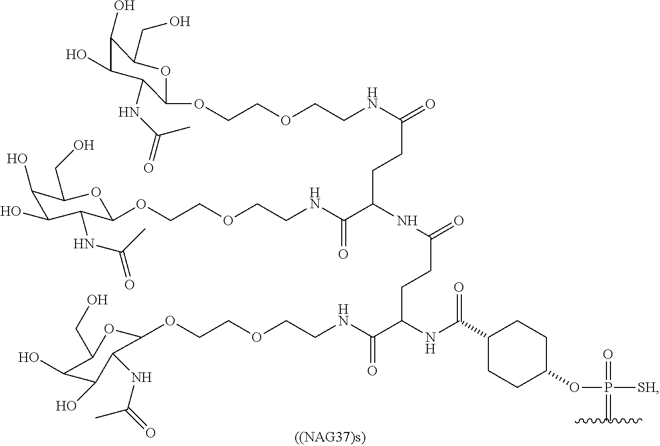

stereoisomer thereof, or a pharmaceutically acceptable salt thereof.

Embodiment 54: The use of Embodiment 53, wherein the dose is a single dose.

Embodiment 55: The use of Embodiment 53, wherein the dose is repeated once, e.g., at an interval of about 28 days (i.e., 4 weeks), about 12 weeks, or about 3 months, between doses, for example about 100 mg every 4 weeks, or about 200 mg every 12 weeks, optionally for about 52 weeks or about 1 year. Embodiment 55a: The use of Embodiment 53, wherein the administration is about 100 mg every 4 weeks for 52 weeks. Embodiment 55b: The use of Embodiment 53, wherein the administration is about 200 mg every 12 weeks for 52 weeks.

Embodiment 56: The use of any one of Embodiments 53-55, wherein the dose is about 50 mg to about 100 mg, for example about 50 mg.

Embodiment 57: The use of any one of Embodiments 53-55, wherein the dose is about 100 mg to about 200 mg, for example about 100 mg.

Embodiment 58: The use of any one of Embodiments 53-55, wherein the dose is about 50 mg to about 200 mg, for example about 200 mg.

Embodiment 59: The use of any one of Embodiments 53-58, wherein the compound is in a sodium salt form.

Embodiment 60: The use of any one of Embodiments 53-59, wherein the compound is used by an injection, for example at a concentration of 200 mg/ml.

Embodiment 61: The use of Embodiment 60, wherein the injection is subcutaneous.

Embodiment 62: The use of any one of Embodiments 53-61, wherein the compound is in a unit dosage form.

Embodiment 63: The use of any one of Embodiments 53-62, wherein the non-alcoholic fatty liver disease is non-alcoholic steatohepatitis (NASH), e.g., pre-cirrhotic non-alcoholic steatohepatitis, NASH-F3, NASH with bridging fibrosis, or biopsy-proven NASH.

Embodiment 64: The use of any one of Embodiments 53-63, wherein the treatment is to a subject having a HSD17β13 rs72613567 mutation, PNPLA3 rs738409 (I148M) mutation, or a combination thereof.

Embodiment 65: The use of any one of Embodiments 53-64, wherein the double-stranded oligonucleotide comprises an antisense strand that comprises a modified nucleotide sequence that differs by 0 or 1 nucleotides from one of the following sequences (5′→3′):

(nucleotide sequence SEQ ID NO: 1)
usCfsasUfcUfaucagAfcUfuCfuUfaCfsg;
(nucleotide sequence SEQ ID NO: 5)
usCfsasUfcUfaUfcAfgAfcUfuCfuUfaCfsg;
(nucleotide sequence SEQ ID NO: 6)
usGfsasUfcCfaAfaAfaUfgUfcCfuAfgGfsc;
or
(nucleotide sequence SEQ ID NO: 7)
usGfsasUfcCfaaaaaUfgUfcCfuAfgGfsc;

and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

Embodiment 66: The use of any one of Embodiments 53-65, wherein the double-stranded oligonucleotide comprises a sense strand that comprises a modified nucleotide sequence that differs by 0 or 1 nucleotides from one of the following sequences (5′→3′):

(nucleotide sequence SEQ ID NO: 3)
cguaagaaGfuCfuGfauagauga;
(nucleotide sequence SEQ ID NO: 8)
cguaagaaGfUfCfugauagauga;
(nucleotide sequence SEQ ID NO: 9)
gccuaggaCfAfUfuuuugiauca;
or
(nucleotide sequence SEQ ID NO: 10)
gccuaggaCfaUfuUfuugiauca;

and wherein a, c, g, i, and u represent 2′-O-methyl adenosine, 2′-O-methyl cytidine, 2′-O-methyl guanosine, 2′-O-methyl inosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

Embodiment 67: The use of any one of Embodiments 53-66, wherein the double-stranded oligonucleotide comprises an antisense strand that comprises a sequence: usCfsasUfcUfaucagAfcUfuCfuUfaCfsg (nucleotide sequence SEQ ID NO:1), and a sense strand that comprises a sequence: (NAG37)s(invAb)scguaagaaGfuCfuGfauagaugas(invAb) (nucleotide sequence SEQ ID NO:11),
and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; s represents a phosphorothioate linkage; and (invAb) represents an inverted abasic deoxyribose residue.

Embodiment 68: The use of any one of Embodiments 53-67, wherein the use reduces the expression of HSD17β13 mRNA by at least about 50% (e.g., at least about 62%, at least about 79%, at least about 84%, at least about 90%, or at least about 96%) compared with that prior to the use, as measured by quantitative Reverse Transcriptase Polymerase Chain Reaction (qRT-PCR) with a liver biopsy sample, for example about day 71 or 113 from the first use.

Embodiment 69: The use of any one of Embodiments 53-68, wherein the use reduces the expression of HSD17β13 protein by at least about 30% (e.g., at least about 50%, at least about 60%, at least about 83%, at least about 92%, or at least about 97%) compared with that prior to the use, as measured by Western Blot with a liver biopsy sample, for example about day 71 or 113 from the first use.

Embodiment 70: The use of any one of Embodiments 53-69, wherein the use reduces ALT (alanine aminotransferase) level by at least about 7.7% (e.g., at least about 14%, at least about 26%, at least about 36%, or at least about 39%), or at least about 40% (e.g., at least about 51%, at least about 53%, or at least about 60%) compared with that prior to the use, as measured in serum, for example about day 71 or 113 from the first use.

Embodiment 71: The use of any one of Embodiments 53-70, wherein the use reduces AST (aspartate aminotransferase) level by at least about 7.5% (e.g., at least about 15%), or at least about 20% (e.g., at least about 24%, or at least about 28%) compared with that prior to the use, as measured in serum, for example about day 71 or 113 from the first use.

Embodiment 72: The use of any one of Embodiments 53-71, wherein the use reduces or slows an increase of liver fat fraction (e.g., a reduction of at least 4-41% or at least 7-8%) compared with that prior to the use, as measured by magnetic resonance imaging, for example about day 71 or 113 from the first use.

Embodiment 73: The use of any one of Embodiments 53-72, wherein the use reduces or slows an increase of liver stiffness (e.g., a reduction of at least 4-37%) compared with that prior to the use, as measured by transient elastography, for example about day 71 or 113 from the first use.

Embodiment 74: The use of any one of Embodiments 53-73, wherein the chemical structure of the ligand comprises:

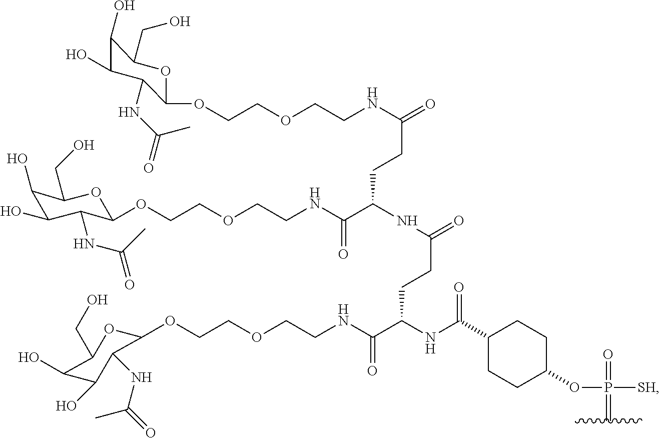

Embodiment 75: The use of Embodiment 74, wherein the compound is in a free acid form having a chemical structure as illustrated in FIG. 5A to 5D.

Embodiment 76: The use of any one of Embodiments 53-73, wherein the chemical structure of the ligand comprises:

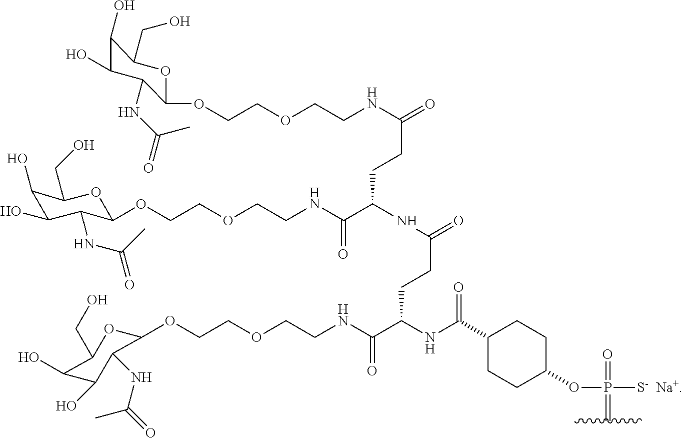

Embodiment 77: The use of Embodiment 76, wherein the compound is in a sodium salt form having a chemical structure as illustrated in FIG. 6A-6D.

Embodiment 78: The use of any one of Embodiments 53-77, wherein the use reduces GGT (gamma-glutamyl transferase) level by at least about 1.4% or at least 8.3% or at least 9%, compared with that prior to the use, as measured in serum, for example at about day 71 or 113 from the first use.

In some cases, one or more human subjects treated with a method described herein achieve=>1 stage (i.e., equal to or greater than 1 stage) improvement in histological fibrosis with no worsening of NASH, for example at week 52 from the start of the treatment. In some instances, improvement in histological fibrosis is assessed with Clinical Research Network (CRN) Scoring. In some instances, no worsening of NASH is defined as no increase in the NAFLD Activity Score (NAS) for steatosis, ballooning, or inflammation.

In some cases, one or more human subjects treated with a method described herein achieve NASH resolution with no worsening of fibrosis, for example at week 52 from the start of the treatment. In some instances, NASH resolution is defined as a ballooning score of 0 and an inflammation score of 0-1. In some instances, no worsening of fibrosis is defined as no increase in CRN fibrosis score.

In some cases, one or more human subjects treated with a method described herein achieve ≥30% relative reduction in liver fat from baseline at week 24 and week 52 from the start of the treatment, for example measured by MRI-PDFF.

### EXAMPLES

### Example 1. Synthesis and Formulation of HSD17B13 RNAi Agents

The compounds of RNAi agents suitable for use herein can be synthesized using standard phosphoramidite technology on solid phase oligonucleotide synthesis as is known in the art. Commercially available oligonucleotide synthesizers (e.g., MerMade96E® (Bioautomation) or MerMade12® (Bioautomation)) may be used. Syntheses can be performed on a solid support made of controlled pore glass (CPG, 500 Å or 600 Å, obtained from Prime Synthesis, Aston, PA, USA). The monomer positioned at the 3′ end of the respective strand may be attached to the solid support as a starting point for synthesis. All 2′-modified RNA phosphoramidites, and inverted abasic phosphoramidites can be purchased commercially. Ligand-containing phosphoramidites can be synthesized that are suitable for addition to the 5′ end of the sense strand. Standard cleavage, deprotection, purification, and annealing steps can be utilized as is known in the art. Further description related to the synthesis of HSD17B13 RNAi agents may be found, for example, in International Patent Application Publication No. WO 2020/061177 (Patent Application No. PCT/US2019/051707) and WO 2018/044350 (PCT/US2017/021147), each of which is incorporated by reference herein in its entirety. HSD17B13 RNAi agents can then be formulated by dissolving in suitable pharmaceutically acceptable excipients.

### Example 2. Phase 1/2a Study to Evaluate the Safety, Tolerability, Pharmacokinetics, and Pharmacodynamic Effects of HSD17B13 RNAi Agent in Normal Healthy Volunteers as Well as in Patients with NASH or NAFLD

The test compound exemplified herein reduces expression of HSD17β13 messenger RNA (mRNA) in hepatocytes. Its sequences are shown in Table 4, and physical properties are listed in Table 5:

TABLE 4
Test Compound
Sense and Antisense Strands (The sense and
antisense strands are annealed to form a
duplex):

| Sense Strand | (NAG37)s(invAb)scguaagaaG | (SEQ ID |
|---|---|---|
| (Modified | fuCfuGfauagaugas(invAb) | NO: 11) |
| Sequence) | | |
| (5′→3′): | | |
| Antisense | usCfsas UfcUfaucagAfcUfuC | (SEQ ID |
| Strand | fuUfaCfsg | NO: 1) |
| (Modified | | |
| Sequence) | | |
| (5′→3′): | | |

TABLE 5
Properties of the Test Compound

| | |
|---|---|
| Chemical Formula: | C₄₉₃H₆₁₀F₁₁N₁₆₃Na₄₃O₃₁₁P₄₃S₇ (Na⁺ form) |
| | C₄₉₃H₆₅₃F₁₁N₁₆₃O₃₁₁P₄₃S₇ (H⁺ form) |
| Molecular Weight: | 16548.9 Da (Na+ form) |
| | 15309.7 Da (H+ form) |
| Physical Appearance: | White to Off-white Powder |

Full chemical structure representations are shown in FIG. 5A to 5D (free acid form) and FIG. 6A to 6D (sodium salt form).

This clinical study evaluated the safety, tolerability, and pharmacodynamics of the test compound in 32 normal healthy volunteers (NHVs) and 18 patients with NASH, or suspected NASH (e.g., non-alcoholic fatty liver disease [NAFLD] with high alanine aminotransferase [ALT]). Double-blind NHV cohorts received single doses of the test compound in 25 mg, 50 mg, 100 mg, or 200 mg or placebo subcutaneously (SC) on Day 1. Open-label patient cohorts received the test compound 25 mg, 100 mg, or 200 mg SC on Days 1 and 29. Liver biopsy was performed in patients predose and Day 71 to evaluate expression levels of HSD17β13 mRNA and protein.

In both NHVs and patients, the test compound treatment was well tolerated, with no treatment-related serious adverse events (SAEs) or drug discontinuations in the study. The most frequently reported treatment-emergent adverse events (TEAEs) were mild injection site reactions that were mild in severity and short in duration. Reductions in hepatic HSD17β13 mRNA were observed following 25 mg (−57%), 100 mg (−86%), and 200 mg (−93%) doses at Day 71. Hepatic HSD17β13 protein levels were similarly reduced at all dose levels. In patients mean maximal decreases in ALT and AST ranged from −42% to −44% and −24% to −28%, respectively, at doses≥100 mg.

The test compound was well tolerated at doses up to 200 mg given on Day 1 (NHVs) or Days 1 and 29 (patients with NASH or NAFLD), with no treatment-related SAEs or drug discontinuations in the study. The incidence of TEAEs was similar in the test compound and placebo groups in NHVs and remained consistent overall in patient groups. This proof-of-concept study demonstrated that the test compound reduces in liver HSD17β13 mRNA and protein, which is accompanied by reductions in ALT and AST.

### Methods

### Participants and Study Design

This was a multicenter, Phase 1/2a clinical study to evaluate the safety, tolerability, pharmacokinetics, and pharmacodynamic effects of injection of the test compound in normal healthy volunteers (NHVs) and patients with NASH or suspected NASH (e.g., NAFLD with high alanine aminotransferase [ALT]). NAFLD criteria was based on magnetic resonance imaging proton density fat fraction (MRI-PDFF)>8% and ALT>upper limit of normal (ULN) (ULN=30 U/L for men and 19 U/L for women). The study was conducted in adult males and females, ages 18 through 55 years, with body mass index (BMI)<35 kg/m² (NHVs) or 19 through 65 years of age with BMI≤40.0 kg/m² (patients with NASH or NAFLD).

Genotyping was performed at screening in patients with NASH or NAFLD to determine mutation status for HSD17β13 rs72613567 and patatin like phospholipase domain containing 3 (PNPLA3) rs738409 (I148M) variants. Individuals were analyzed by HSD17β13 rs726131567 genotypes into T/T (homozygous wild type, or no mutation) or T/TA (heterozygous mutation) groups. Individuals who were homozygotes for rs726131567:TA were excluded from study. Similarly, individuals were analyzed by PNPLA3 genotypes into C/C (homozygous wild-type, or no mutation), C/G (heterozygous mutation), or G/G (homozygous mutation) groups.

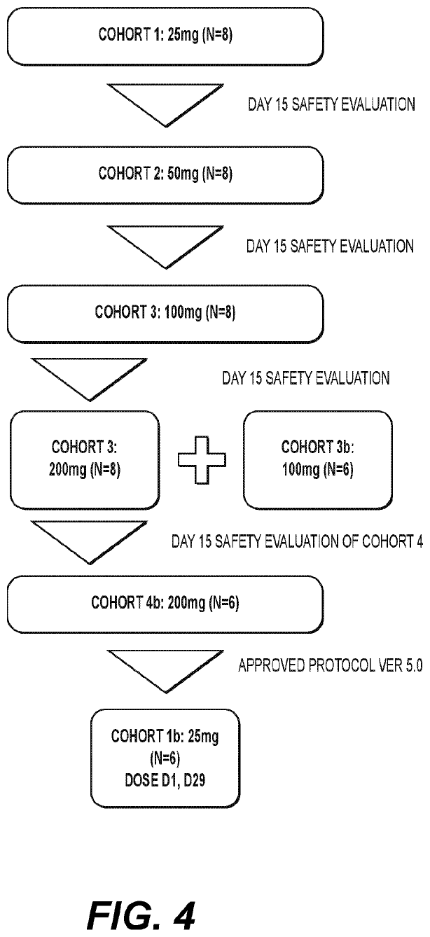

Subjects enrolled into the study in a double-blind (NHV Cohorts 1 [25 mg], 2 [50 mg], 3 [100 mg], 4 [200 mg]) or open-label (patient Cohorts 1b [25 mg], 3b [100 mg], 4b [200 mg]) fashion (Table 6). NHV subjects were randomized at a ratio of 4:4 (active:placebo) to receive a single subcutaneous (SC) injection of either the test compound (25, 50, 100, or 200 mg) or placebo (Cohorts 1 through 4) on Day 1. Patients with NASH or NAFLD received open-label the test compound (25, 100, or 200 mg in Cohorts 1b, 3b, and 4b, respectively) on Days 1 and 29. The study design is summarized in FIG. 4 and Table 6.

TABLE 6
Cohort Summary

| Cohort | Population | Blinding | # Subjects | Dosing Schedule |
|---|---|---|---|---|
| 1 | NHVs | Double blind | 8 (4 active: 4 placebo) | 25 mg Day 1 only |
| 1b | Patients with | Open Label | up to 6 (all active) | 25 mg Days 1, 29 |
| | NAFLD (biopsies on | | | |
| | screen and day 71) | | | |
| 2 | NHVs | Double blind | 8 (4 active: 4 placebo) | 50 mg Day 1 only |
| 3 | NHVs | Double blind | 8 (4 active: 4 placebo) | ≤100 mg Day 1 only |
| 4 | NHVs | Double blind | 8 (4 active: 4 placebo) | ≤200 mg Day 1 only |
| 3b | Patients with | Open Label | up to 6 (all active) | ≤100 mg Days 1, 29 |
| | NAFLD (biopsies on | | | |
| | screen and day 71) | | | |
| 4b | Patients with | Open Label | up to 6 (all active) | ≤200 mg Days 1, 29 |
| | NAFLD (biopsies on | | | |
| | screen and day 71) | | | |

NASH = non-alcoholic steatohepatitis; NHVs = normal healthy volunteers.

For Cohorts 1 through 4, the test compound or placebo was administered to 2 sentinel subjects (1 the test compound, 1 placebo). Following the Day 3 evaluation in these subjects, the remaining subjects in the cohort were treated, staggered by at least 30 minutes such that no 2 subjects were dosed simultaneously. Cohorts 1 through 4 were enrolled sequentially with patient cohorts opening for enrollment in a step-wise manner. Sentinel dosing was not required in patient cohorts. Dose levels by cohort are outlined in Table 6. NHVs remained at the clinical facility for 3 days for dose administration on Day 1 (Cohorts 1, 2, 3, and 4). NHVs returned to the clinical facility for outpatient visits. Patients in Cohorts 1b, 3b, and 4b were at the clinical facility for approximately 4 hours (2 hours before dosing and 2 hours after dosing) on dosing days (Days 1 and 29).

### Study Treatments

Fifty subjects (32 NHVs and 18 patients) were enrolled. Of the 58 NHVs screened, 8 were screen failed (mostly due to not meeting inclusion or exclusion criteria) and 18 were not assigned (due to a lapse in 45-day screening period because of the COVID-19 global pandemic). For NHV Cohorts 1 through 4, each double-blind cohort enrolled 8 subjects (4 active:4 placebo). All NHV cohorts received single doses of the test compound or placebo at dose levels of 25 mg (Cohort 1), 50 mg (Cohort 2), 100 mg (Cohort 3), and 200 mg (Cohort 4) on Day 1.

Among the 34 patients with NASH or NAFLD that were screened, 16 were screen failed (due to not meeting inclusion or exclusion criteria) (Table 6). For the 18 patients who entered the open-label Cohorts 1b, 3b, and 4b, 6 were enrolled per cohort. All patient cohorts received the test compound at dose levels of 25 mg (Cohort 1b), 100 mg (Cohort 3b), or 200 mg (Cohort 4b) on Days 1 and 29. Patients were followed until Day 113 (Week 16).

### Study Assessments and Procedures

Inpatients with NASH or NAFLD, pharmacodynamic effects of the test compound were measured by hepatic HSD17β13 mRNA (qRT-PCR) and protein (Western Blot) expression, taken from paired liver biopsies at screening and Day 71 to measure the activity of the test compound against HSD17(313. For gene expression measurements by qRT-PCR, HSD17B13 and endogenous control, beta-actin (ACTB) and mRNA were measured in both Day 71 and screening biopsies. Relative expression was calculated using the 2(−delta C[T]) method of normalization. Changes to HSD17B13 protein expression were assessed as the ratios of HSD17B13/Vinculin protein at D71 to pre-dose baseline levels. Markers of liver injury, such as ALT and AST, were measured from screening to Day 113. Liver imaging was performed to assess changes in liver fat fraction using MRI-PDFF, and liver stiffness using transient elastography (FibroScan, kPa) at screening and Day 71. HSD17B13 rs72613567 and patatin like phospholipase domain containing 3 (PNPLA3) rs738409 single nucleotide polymorphisms (SNPs) were detected in whole blood by real-time PCR, and results were confirmed using Sanger sequencing at screening.

Safety assessments included determining the incidence of adverse events (AEs)/serious adverse events (SAEs), conducting physical examinations, and recording vital sign measurements (blood pressure, heart rate, temperature, and respiratory rate), electrocardiograms (ECGs), clinical laboratory tests (blood and urine), concomitant medications/therapy, and recording reasons for treatment discontinuation in both NHV and patient cohorts.

### Statistical Analysis

All safety results, demographic and baseline characteristics data, and pharmacodynamic results were summarized based on data collected from all enrolled subjects who received at least one dose of active drug or placebo. Treatment-emergent adverse events were coded using the Medical Dictionary for Regulatory Activities (MedDRA, version 24.0).

For analyses of pharmacodynamic parameters, descriptive statistics summaries and derived knockdown data from baseline were provided for HSD17β13 mRNA and protein. Non-parametric Wilcoxon signed-rank test was performed to test the significance of change from baseline at Day 71. Also, for liver enzyme parameters such as ALT and AST, descriptive statistics summaries were provided along with derived change from baseline and percent change from baseline summaries. Non-parametric Wilcoxon signed-rank test was also performed to test the significance of change from baseline at Days 29, 71 and 113 of ALT and AST. For hepatic imaging (MRI-PDFF and FibroScan), descriptive statistics summaries and derived percent change from baseline were provided. Baseline values were defined as the last non-missing observation (predose value closest to the first dose) for each subject prior to the dosing of study medication (i.e., start of injection on Day 1). The data were analyzed using SAS software, version 9.4, and figures were generated using GraphPad Prism software, version 8.3.0.

### Study Oversight

The ethics committee at each participating center approved the protocol. The study was conducted in accordance with the principles of the Declaration of Helsinki, the International Conference on Harmonisation Good Clinical Practice guidelines, and applicable regulatory requirements. All subjects provided written informed consent before enrollment.

### Results

### Patient Characteristics and Disposition

Healthy volunteers' demographic characteristics are shown in Table 7. All enrolled NHVs completed all planned assessments.

TABLE 7
Summary of Demographics - Healthy Volunteer Cohorts

| | Cohort 1 | | Cohort 2 | | Cohort 3 | | Cohort 4 | |
|---|---|---|---|---|---|---|---|---|
| | (25 mg, | | (50 mg, | | (100 mg, | | (200 mg, | |
| | Day 1) | | Day 1) | | Day 1) | | Day 1) | |
| Parameter; Statistic | (N = 4) | | (N = 4) | | (N = 4) | | (N = 4) | |
| Age (years); Mean (min, max) | 23.5 | (19, 31) | 31.0 | (24, 50) | 32.3 | (20, 45) | 33.0 | (22, 41) |
| Male; n (%) | 3 | (75.0%) | 2 | (50.0%) | 3 | (75.0%) | 1 | (25.0%) |

Ethnicity; n (%)

| Hispanic or Latino | 0 | | 0 | | 2 | (50.0%) | 0 | |
|---|---|---|---|---|---|---|---|---|
| Not Hispanic or Latino | 4 | (100.0%) | 3 | (75.0%) | 2 | (50.0%) | 4 | (100.0%) |

| | | | | | |
|---|---|---|---|---|---|
| Not Reported | 0 | 1 | (25.0%) | 0 | 0 |

Race; n (%)

| Asian | 2 | (50.0%) | 0 | | 1 | (25.0%) | 1 | (25.0%) |
|---|---|---|---|---|---|---|---|---|
| Native Hawaiian or Other | 0 | | 0 | | 0 | | 1 | (25.0%) |
| Pacific Islander | | | | | | | | |
| White | 2 | (50.0%) | 3 | (75.0%) | 2 | (50.0%) | 2 | (50.0%) |

| | | | | | |
|---|---|---|---|---|---|
| Other | 0 | 0 | 1 | (25.0%) | 0 |

| | | | | | |
|---|---|---|---|---|---|
| More than One Race Reported | 0 | 1 | (25.0%) | 0 | 0 |

| | | | | |
|---|---|---|---|---|
| Weight (kg); Mean (min, max) | 63.2 (53.2, | 72.8 (58.3, | 76.1 (64.0, | 78.5 (56.5, |
| | 68.8) | 88.6) | 89.1) | 93.0) |
| BMI (kg/cm2); Mean (min, max) | 22.55 (21.9, | 23.78 (20.2, | 24.98 (20.6, | 28.40 (21.0, |
| | 24.1) | 27.3) | 28.1) | 31.5) |

BMI = body mass index.

Patients with NASH or NAFLD: Mean (SD) age was 44.5 (9.9) years. Four subjects (22.2%) were female, and 14 (77.8%) subjects were male. Thirteen (72.2%) subjects were Asian, 3 subjects (16.7%) were White, 1 subject (5.6%) was Black or African American, and 1 subject (5.6%) was other. At baseline, the range of MRI-PDFF was 10.34% to 30.73% and the range of ALT was 28 U/L (for a female subject) to 144 U/L. Suspected NASH (e.g., NAFLD with high ALT) was defined by MRI-PDFF>8% with ALT>ULN (ULN=30 U/L for men and 19 U/L for women. Alternatively, biopsy confirmed NASH within 1 year of screen visit was acceptable for inclusion.) These patients presented with typical NAFLD comorbidities, with 50% (n=9), 44.4% (n=8), and 44.4% (n=8) having hyperlipidemia, type 2 diabetes mellitus, and hypertension, respectively.

A summary of genotyping results for NAFLD patients by cohort is provided in Table 8. For the HSD17β13 rs72613567 variant, 12 subjects (67%) had no mutation detected (y/T), and 6 subjects (33%) were heterozygotes (5/TA). For the PNPLA3 rs738409 variant, 11 subjects (61%) had no mutation detected (C/C), 4 subjects (22%) were heterozygotes (C/G), and 3 subjects (17%) were homozygotes (G/G). Baseline, concomitant medications taken by patients with NASH or NAFLD in the clinical study included statins (HMG COA reductase inhibitors) (39% of subjects), biguanides (metformin) (33% of subjects), ACE inhibitors (28% of subjects), angiotensin II receptor blockers (22% of subjects), calcium channel blockers (dihydropyridine derivates) (22% of subjects), glucagon-like peptide-1 (GLP-1) analogues (5.6% of subjects), and sodium-glucose co-transporter 2 (SGLT2) inhibitors (5.6% of subjects). All subjects completed the study without early termination.

TABLE 8
Summary of Demographics and Medical History for Patients with NASH or NAFLD

| | | | |
|---|---|---|---|
| | Cohort 1b | Cohort 3b | Cohort 4b |
| | (25 mg, | (100 mg, | (200 mg, |
| | Day 1, 29) | Day 1, 29) | Day 1, 29) |
| Parameter; Statistic | (N = 6) | (N = 6) | (N = 6) |

Demographics

| Age (years), mean (min, max) | 46 | (32, 56) | 45 | (40, 50) | 43 | (22, 61) |
|---|---|---|---|---|---|---|
| Male (%) | 5 | (83%) | 5 | (83%) | 4 | (67%) |
| Ethnicity - n (%) | | | | | | |
| Not Hispanic or Latino | 6 | (100.0) | 6 | (100.0) | 6 | (100.0) |
| Race - n (%) | | | | | | |
| Asian | 4 | (66.7) | 5 | (83.3) | 4 | (66.7) |
| Native Hawaiian or Other Pacific | 0 | | 1 | (16.7) | 0 | |
| Islander | | | | | | |
| White | 2 | (33.3) | 0 | | 1 | (16.7) |
| Other | 0 | | 0 | | 1 | (16.7) |
| Weight (kg), mean (min, max) | 96.4 | (68, 119.7) | 87.2 | (65.8, 115.7) | 97.5 | (74, 117.4) |
| BMI (kg/cm²), mean (min, max) | 32.17 | (25.7, 41.1) | 29.1 | (25.1, 36.5) | 33.7 | (28.2, 39.2) |
| Baseline laboratory | | | | | | |
| results, ALT and AST | | | | | | |
| Baseline ALT (U/L), mean (min, | 45.7 | (28, 69) | 68.3 | (30, 144) | 76 | (28, 100) |
| max) | | | | | | |
| Baseline AST (U/L), mean (min, | 21.7 | (17, 27) | 33.5 | (22, 63) | 50.3 | (18, 131) |

max)

Genotype

| HSD17B13 rs72613567, n (%) | | | | | | |
|---|---|---|---|---|---|---|
| T/T | 5 | (83) | 4 | (67) | 3 | (50) |
| T/TA | 1 | (17) | 2 | (33) | 3 | (50) |
| PNPLA3 rs738409, n (%) | | | | | | |
| C/C | 3 | (50) | 5 | (83) | 3 | (50) |
| C/G | 2 | (33) | 0 | | 2 | (33) |
| G/G | 1 | (17) | 1 | (17) | 1 | (17) |

ALT = alanine aminotransferase; AST = aspartate aminotransferase; BMI = body mass index; C/C = no mutation; C/G = heterozygous; G/G = homozygous; max = maximum; min = minimum; NASH = non-alcoholic steatohepatitis; SD = Standard Deviation; T/T = no mutation; T/TA = heterozygous.
^{a} Coded using the Medical Dictionary for Regulatory Activities version 23.0.

### Pharmacodynamics

Hepatic HSD17B13 Protein and mRNA

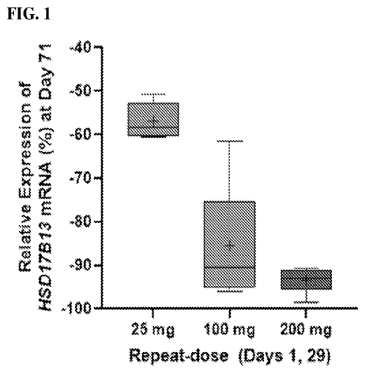

The test compound reduced the hepatic mRNA encoding HSD17β13 protein from the pretreatment baseline by a mean (min-max) value of 56.9% (50.7%-60.5%), 85.5% (61.6%-96.1%) and 93.4% (90.8%-98.6%) in the 25 mg, 100 mg, and 200 mg dose cohorts, respectively, on Day 71 (FIG. 1). The overall pooled subject HSD17β13 mRNA reduction was 78.6% (50.7%-98.6%, p-value<0.0001). In the 200 mg dose cohort, all 6 subjects demonstrated greater than 90% reduction in hepatic expression of HSD17β13 mRNA. The Hepatic HSD17β13 protein levels were similarly reduced by a mean of >34%, >86%, and 83% in the 25 mg, 100 mg, and 200 mg dose cohorts, respectively, and average change from baseline in HSD17β13 protein from pooled cohorts was 63% (range from −97.8% to 53.8%, p=0.0017), with multiple measurements (for 3 patients at baseline and 6 patients on Day 71) that were below the assay's level of quantitation (Table 9).

TABLE 9
Summary of HSD17β13 mRNA and Protein Knockdown
of the Test Compound in Patients with NASH or NAFLD

| | % Change from baseline Mean (min, max) | | | |
|---|---|---|---|---|
| | 25 mg | 100 mg | 200 mg | |
| | (N = 6) | (N = 6) | (N = 6) | Pooled |
| Hepatic HSD17β13 | −56.9% | −85.5% | −93.4% | −78.6% |
| mRNA at Day 71 | (−60.5%, −50.7%) | (−96.1%, −61.6%) | (−98.6%, −90.8%) | (−98.6%, −50.7%) |
| | | | | p < 0.0001 |
| Hepatic HSD17β13 | <−33.8% | <−86% | −83% | −63.2% |
| Protein at Day 71 ^{a} | (<−92.4%, 53.8%) | (−97.8%, <−63.0%) | (−85.2%, −80.8%) ^{b} | (−97.8%, 53.8%) |
| | | | | p = 0.0017 |

LLOQ = lower limit of quantitation; mRNA = messenger RNA; NASH = non-alcoholic steatohepatitis.
^{a} Several patients had HSD17β13 protein levels at D 71 below LLOQ, in which case LLOQ was used for calculation of means.
^{b} n = 2 (3 samples with baseline HSD17β13 below LLOQ, 1 sample failed assay acceptance criteria).

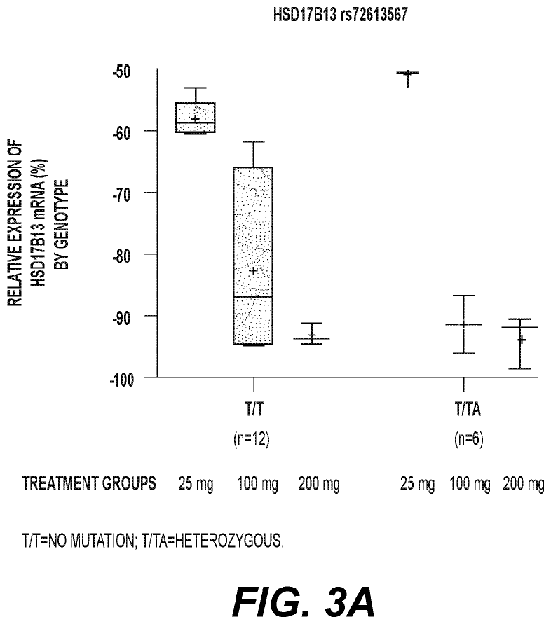

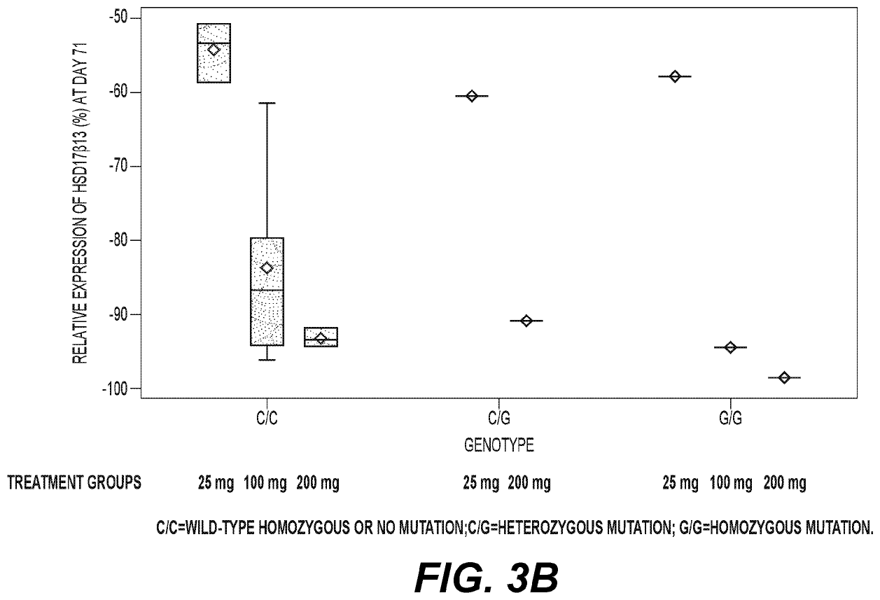

The observed data for HSD17B13 mRNA reduction suggest that robust on-target pharmacological responses were achieved in the liver, with trends toward dose dependency at 100 and 200 mg dose levels, and clear dose dependency at 25 and 100 mg dose levels. Observed pharmacodynamic effects were not affected by HSD17β13 (rs72613567, T>TA) and/or PNPLA3 (rs738409, C>G) mutations. At each separate dose level, a similar reduction in HSD17β13 mRNA was observed for patients who had no mutation compared with patients who were heterozygotes for rs72613567 splice variant (FIG. 3A). Meanwhile, at each dose level, a similar reduction in HSD17β13 mRNA was observed for patients who had no mutation compared with patients who were heterozygotes or homozygotes for PNPLA3 rs738409 polymorphism (FIG. 3B). Thus, HSD17β13 knockdown was consistent in this study regardless of genotype or mutation status.

### ALT, AST, and GGT

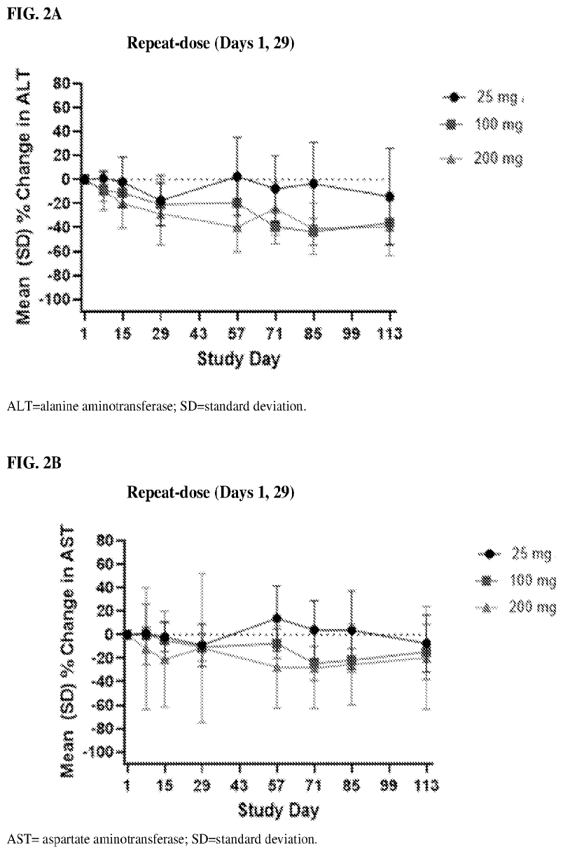

In patients with NASH or NAFLD, serum biomarkers of liver injury decreased following treatment with the test compound, demonstrating reduction in ALT between the 25 and 100 mg dose levels. The 100 and 200 mg dose levels showed similar reductions in ALT. The mean percent change from baseline at Day 71 (same time as biopsy) for ALT ranged from −7.7% (25 mg dose group) to −39.3% (100 mg) and −42.2% (200 mg) (P<0.001 for pooled cohorts) (FIG. 2A). At Day 113, or by the end of study, the mean percent change from baseline for ALT was −14%, −36%, and −39% for the 25, 100, and 200 mg dose levels, respectively (P<0.01 for pooled cohorts) (FIG. 2A). Similar to ALT, AST was reduced between the 25 and 100 mg dose levels, with similar reductions observed between the 100 and 200 mg doses. The mean percent change from baseline at Day 71 for AST was 4%, −24%, and −28% for the 25, 100, and 200 mg dose levels, respectively (P<0.01 for pooled cohorts) (FIG. 2B). At Day 113, mean percent change from baseline for AST was −7.5%, −14.7% and −19.7% for the 25, 100, and 200 mg dose levels, respectively (FIG. 2B). Two doses of 25, 100, or 200 mg of the test compound were given to patients at Day 1 and Day 29 and safety and pharmacodynamic parameters were measured through end of study, or Day 113. Duration of ALT reduction was sustained through Day 85, or 8 weeks post-last dose, for 100 and 200 mg dose levels. Duration of AST reduction was sustained through Day 71, or 6 weeks post-dose, for 100 and 200 mg dose levels. ***P<0.001; **P<0.01, *<01 are p-values of pooled cohorts (25, 100 and 200 mg dose levels at specified timepoints using non-parametric testing (Wilcoxon signed rank test)). Mean body weight was stable for the duration of the study within each dose group. Two subjects lost weight during the study and showed the largest percent decrease in ALT from baseline within their dose groups. These subjects had a decrease in body weight by 4.6 kg (25 mg dose group) and 2.2 kg (100 mg group) by Day 71 corresponding to 60% and 51% reductions in ALT, respectively. All other subjects maintained stable body weight throughout the study; therefore, all other decreases in liver enzymes were observed independent of changes in body weight. GGT level was reduced by about: 1.4%, 8.3%, and 9% in the patient cohorts of 25 mg, 100 mg, and 250 mg respectively, compared with that prior to the administration, as measured in serum.

### Liver Fat Fraction and Liver Stiffness

The mean relative changes from baseline to Day 71 in liver fat fraction by MRI-PDFF were 14.4%, −7.6%, and −7.3% at doses 25, 100, and 200 mg, respectively (Table 10).

TABLE 10
Summary of Liver Fat Fraction (MRI-PDFF) and Liver Stiffness
(FibroScan) in Patients with NASH or NAFLD

| % Change from baseline Mean (min, max) | | |
|---|---|---|
| 25 mg | 100 mg | 200 mg |
| (N = 6) | (N = 6) | (N = 6) |

Liver fat fraction

| | | | |
|---|---|---|---|
| Baseline | 17.25 | 16.95 | 25.42 |
| | (13.85, 19.95) | (10.34, 25.17) | (15.10, 30.73) |
| Day 71 | 14.41% | −7.56% | −7.33% |
| | (−36.0%, 87.9%) | (−40.7%, 23.6%) | (−24.1%, 5.9%) |

Liver stiffness (kPa)

| | | | |
|---|---|---|---|
| Baseline | 5.47 | 8.42 | 8.30 |
| | (3.8, 9.9) | (5.3, 13.9) | (7.3, 9.0) |
| Day 71 | 16.70% | 2.18% | 4.18% |
| | (−5.3%, 37.5%) | (−16.5%, 33.9%) | (−36.8%, 54.4%) |

kPa = kilopascal;
MRI-PDFF = magnetic resonance imaging proton density fat fraction

The mean percent change from baseline for liver stiffness at Day 71 (in kPa) was +16.7%, +2.2%, and +4.2% at doses 25, 100, and 200 mg, respectively (Table 10).

At both 100 mg and 200 mg dose levels, there was a less pronounced increase in liver stiffness, and a decrease in liver fat fraction compared to the 25 mg dose level as indicated by mean change from baseline at Day 71.

### Safety

The test compound was well tolerated in both NHVs and patients with NASH or NAFLD (Table 11 and Table 12), with no AE-related study or drug discontinuations, no dose-limiting toxicities, or deaths. There was no recurrent pattern of adverse laboratory findings indicative of end organ toxicity. The most frequently reported TEAEs were injection site reactions (injection site bruising, injection site erythema, and injection site swelling) that were mild in severity and short in duration. These events occurred in 5 of 16 NHVs who received the test compound and no patients with NASH or NAFLD. The remaining TEAEs were those commonly observed in clinical studies (Table 12). A single treatment-emergent SAE of soft tissue injury that required hospitalization was reported in the 200 mg cohort. The subject was admitted for a right arm soft tissue injury following an accident that occurred at work (unrelated to the time and site of injection) and was discharged from the hospital 2 days later. The subject recovered from the event and continued in the study. The SAE was considered not related to study drug.

### CONCLUSION

The test compound was well tolerated at doses up to 200 mg given once (NHVs) or twice (patients with NASH or NAFLD), with no treatment-related serious adverse events, no pattern of adverse drug related laboratory findings, and no drug discontinuations in the study. The incidence of TEAEs was similar in the test compound and placebo groups in NHVs and remained consistent overall in patient cohorts. There were no mean increases in liver fat, and no adverse changes in biomarkers of liver injury across all dose levels. Reductions in liver HSD17β13 mRNA and protein were observed and corresponded with ALT and AST reductions, which is a clinically meaningful signal of reduced liver injury. AST concentrations were decreased by an average of 7.5%, 24.5%, and 28.3% for the 25, 100, and 200 mg doses, respectively and sustained for 12 weeks with the 100 and 200 mg doses. Similarly, ALT levels were also reduced up to 14.1%, 43.6%, and 42.3% for the 25, 100, and 200 mg doses, respectively and sustained for 12 weeks with the 100 and 200 mg doses on Day 1 and Day 29. The reductions in hepatic HSD17B13 mRNA and HSD17B13 protein demonstrate robust on-target pharmacologic response in the liver with 100 and 200 mg doses. Thus, the data at least support a dosing regimen of 100 mg every 4 weeks (Q4W).

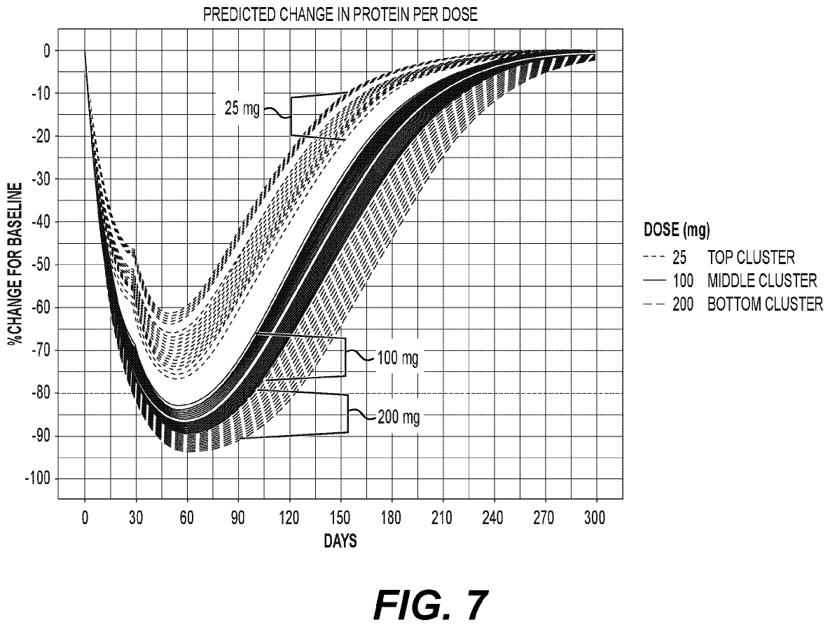

Pharmacokinetics/pharmacodynamics (PK/PD) modelling of HSD17β13 protein knockdown and ALT predict that the 200 mg dose maintains a reduction in HSD17β13 protein and ALT for 90 days, further supporting a test dosing regimen of 200 mg every 3 months or 12 weeks. As shown in FIG. 7, doses of 100 mg and 200 mg maintain the >80% decrease in HSD17β13 protein from baseline for 3 months or longer. As T_{1/2} of the protein is 10.6 days, assuming complete knockdown in 30 days for example about 87% decrease, doses of 200 mg are postulated to induce full knock-down of HSD17β13 protein.

TABLE 11
Overall Summary of Adverse Events (Single-Dose Healthy Volunteer Cohorts)

| | Single Dose (Day 1) | | | | |
|---|---|---|---|---|---|
| | Normal Healthy Volunteers | | | | |
| | Test Compound | | | | |
| | Cohort 1 | Cohort 2 | Cohort 3 | Cohort 4 | |
| | (25 mg, | (50 mg, | (100 mg, | (200 mg, | Pooled |
| | Day 1) | Day 1) | Day 1) | Day 1) | Placebo |
| | (N = 4) | (N = 4) | (N = 4) | (N = 4) | (N = 16) |
| | n (%) # | n (%) # | n (%) # | n (%) # | n (%) # |
| Any AE | 4 (100) 6 | 1 (25.0) 2 | 4 (100) 10 | 3 (75.0) 7 | 10 (62.5) 14 |
| Any TEAE | 4 (100) 6 | 1 (25.0) 2 | 4 (100) 9 | 3 (75.0) 6 | 10 (62.5) 14 |
| Any Treatment-Related TEAE | 0 | 0 | 1 (25.0) 1 | 3 (75.0) 5 | 0 |
| Any Grade 3+ (Severe) TEAE | 0 | 0 | 0 | 0 | 0 |
| Any TEAE at the Injection | 1 (25.0) 1 | 0 | 1 (25.0) 1 | 3 (75.0) 3 | 0 |
| Site | | | | | |
| Any TEAE Related to LISR | 0 | 0 | 0 | 2 (50.0) 2 | 0 |
| Any SAE | 0 | 0 | 0 | 0 | 0 |
| Any AE Resulting in Study | 0 | 0 | 0 | 0 | 0 |
| Drug Withdrawal, Study | | | | | |
| Discontinuation, or Death | | | | | |

| | Repeat Dose (Days 1 and 29) | | | |
|---|---|---|---|---|
| | NASH/NAFLD | | | |
| | Test Compound | | | |
| | Cohort 1b | Cohort 3b | Cohort 4b | |
| | (25 mg, | (100 mg, | (200 mg, | |
| | Day 1, 29) | Day 1, 29) | Day 1, 29) | Overall |
| | (N = 6) | (N = 6) | (N = 6) | (N = 18) |
| | n (%) # | n (%) # | n (%) # | n (%) # |
| Any AE | 3 (50.0) 9 | 2 (33.3) 6 | 4 (66.7) 11 | 9 (50.0) 26 |
| Any TEAE | 3 (50.0) 8 | 2 (33.3) 5 | 4 (66.7) 10 | 9 (50.0) 23 |
| Any Treatment-Related TEAE | 1 (16.7) 1 | 1 (16.7) 2 | 2 (33.3) 4 | 4 (22.2) 7 |
| Any Grade 3+ (Severe) TEAE | 0 | 0 | 0 | 0 |
| Any TEAE at the Injection | 0 | 0 | 0 | 0 |
| Site | | | | |
| Any TEAE Related to LISR | 0 | 0 | 0 | 0 |
| Any SAE | 0 | 0 | 1 (16.7) 1 | 1 (5.6) 1 |
| Any TESAE | 0 | 0 | 1 (16.7) 1 | 1 (5.6) 1 |
| Any Treatment-Related | 0 | 0 | 0 | 0 |
| TESAE | | | | |
| Any AE Resulting in Study | 0 | 0 | 0 | 0 |
| Drug Withdrawal, Study | | | | |
| Discontinuation, or Death | | | | |

AE = adverse event, NA = not applicable; NASH = non-alcoholic steatohepatitis; TEAE = treatment-emergent adverse event, LISR = local injection site reaction, SAE = serious adverse event; TESAE = treatment-emergent serious adverse event.
Note:
Categories are not mutually exclusive. n (%) # denotes the number of subjects, percent of subjects, and number of events in each category, respectively.

TABLE 12
Summary of TEAEs by Preferred Term Reported in 2 or
More Subjects in a Cohort (NHV and NASH or NAFLD)

| | Single Dose (Day 1) | | | | |
|---|---|---|---|---|---|
| | Normal Healthy Volunteers | | | | |
| | Test Compound | | | | |
| | Cohort 1 | Cohort 2 | Cohort 3 | Cohort 4 | Pooled |
| | (25 mg) | (50 mg) | (100 mg) | (200 mg) | Placebo |
| | (N = 4) | (N = 4) | (N = 4) | (N = 4) | (N = 16) |
| Preferred Term (PT) | n (%) # | n (%) # | n (%) # | n (%) # | n (%) # |
| Any TEAE | 4 (100) 6 | 1 (25.0) 2 | 4 (100) 10 | 3 (75.0) 7 | 10 (62.5) 14 |
| Headache | 0 | 0 | 1 (25.0) 1 | 0 | 2 (12.5) 2 |
| Dermatitis | 0 | 0 | 0 | 0 | 2 (12.5) 2 |
| Injection site erythema | 0 | 0 | 0 | 2 (12.5) 2 | 0 |

| | Repeat Dose (Days 1 and 29) | | |
|---|---|---|---|
| | NASH/NAFLD | | |
| | Test Compound | | |
| | Cohort 1b | Cohort 3b | Cohort 4b |
| | (N = 6) | (N = 6) | (N = 6) |
| Preferred Term (PT) | n (%) # | n (%) # | n (%) # |
| Any TEAE | 3 (50.0) 8 | 2 (33.3) 5 | 4 (66.7) 10 |
| TEAE in 2 or more subjects ^{a} | — | — | — |

NASH = non-alcoholic steatohepatitis; NHV = normal healthy volunteer; TEAE = treatment-emergent adverse event.
^{a} All reported TEAEs were single event terms across various system organ classes.
Note:
Coded using the Medical Dictionary for Regulatory Activities version 23.0. Subjects are counted once within each Preferred Term. Events are counted the number of times they occur. n (%) # denotes the number of subjects, percent of subjects, and number of events in each category, respectively.

### Example 3. HSD17B13 Minimization for the Treatment of NASH: A Double-Blind, Placebo-Controlled Phase 2b Study to Evaluate the Efficacy and Safety of HSD17B13 RNAi Agent in Adults with Pre-Cirrhotic Non-Alcoholic Steatohepatitis

This is a double-blind, randomized, placebo controlled, Phase 2b, multi-center study designed to assess efficacy and safety of the test compound (Table 4) in adult patients with NASH and bridging (F3) fibrosis.

The study includes 3 treatment arms including 2 arms receiving the test compound with 2 different dosing regimens and one placebo arm. Following a screening period of up to 70

days, eligible participants are randomized into one of 3 treatment arms:
1) 100 mg of the test compound once every 4 weeks (Q4W) for 52 weeks
2) 200 mg of the test compound once every 12 weeks (Q12W) for 52 weeks, or
3) Placebo once every 4 weeks (Q4W) for 52 weeks.

Approximately 246 participants are planned for study participation. Dose cohorts are enrolled in parallel with approximately 82 participants in each treatment arm and assigned randomly in a 1:1:1 ratio.

### Inclusion Criteria Include, but are not Limited to

Body Mass Index (BMI)≥25 kilogram per meter square (kg/m²) (all ethnic origins) except for Asian participants who qualify for the study with BMI≥23 kg/m² at Screening.
In the opinion of the investigator, there are features of metabolic syndrome and NAFLD is the most likely cause of liver disease.
A liver biopsy at baseline showing NAFLD Activity Score (NAS)>=4 with at least 1 point each in steatosis, inflammation and ballooning and Fibrosis CRN score of 3.
Able and willing to comply with all study assessments, including a liver biopsy at Week 52.

### Exclusion Criteria Include, but are not Limited to:

Cirrhosis (based on screening biopsy or historical biopsy showing definitive cirrhosis).
Current alcohol consumption≥14 standard drinks (24 units, 196 g ethanol) per week for females or ≥21 standard drinks (37 units, 294 g ethanol) per week for males.
Weight reduction surgery (including gastric banding and intragastric balloon insertion) within 2 years of Screening 1.
History of cancer within previous 2 years from Screening 1, except adequately resected non-melanoma skin cancer.

TABLE 13
Primary Outcome Measures

| Outcome Measure | TimeFrame | Description |
|---|---|---|
| Percentage of Participants | At Week 52 | Improvement in histological fibrosis is |
| Achieving =>1 Stage | | assessed with Clinical Research Network |
| Improvement in Histological | | (CRN) Scoring and no worsening of |
| Fibrosis with no Worsening of | | NASH is defined as no increase in the |
| NASH | | NAFLD Activity Score (NAS) for |
| | | steatosis, ballooning, or inflammation. |
| Percentage of Participants | At Week 52 | NASH resolution is defined as a |
| Achieving NASH Resolution | | ballooning score of 0 and an |
| with no Worsening of Fibrosis | | inflammation score of 0-1 and no |
| | | worsening of fibrosis is defined as no |
| | | increase in CRN fibrosis score. |

Secondary outcome measures at least include percentages of participants achieving ≥30% relative reduction in liver fat from baseline using magnetic resonance imaging such as MRI-PDFF at Week 24 and Week 52, change from baseline in Liver stiffness measurement (LSM) by transient elastography such as Vibration-controlled transient elastography (VCTE), and change from Baseline in Alanine Aminotransferase (ALT), Aspartate Aminotransferase (AST) and Gamma-glutamyl transferase (GGT) (Units Per Liter) [Time Frame: Baseline (Day 1) and at Week 24 and 52].

1. A method for treating a non-alcoholic fatty liver disease in a human subject in need thereof, comprising administering to the human subject a compound or a pharmaceutically acceptable salt thereof, at a dose of about 25 to about 200 mg, wherein the dose is calculated based on a free acid form of the compound, wherein the compound comprises a double-stranded oligonucleotide linked to a ligand, wherein the double-stranded oligonucleotide reduces expression of HSD17β13 in the human subject, and wherein the ligand comprises a chemical structure of: 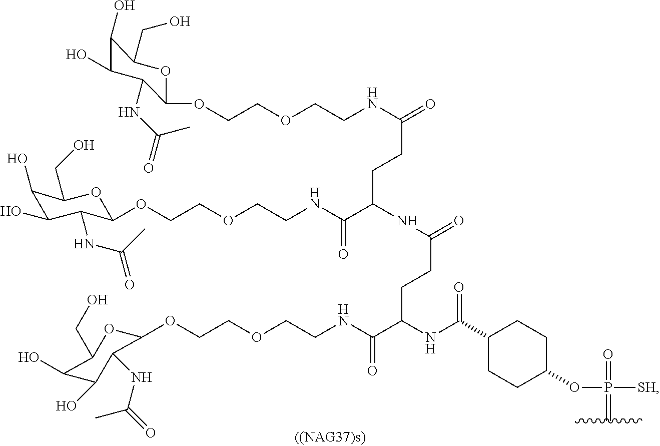
stereoisomer thereof, or a pharmaceutically acceptable salt thereof.

2. The method of claim 1, wherein the administration is a single dose.

3. The method of claim 1, wherein the administration is repeated at an interval of about 28 days, about 12 weeks, or about 3 months, between doses, optionally for about 52 weeks or about 1 year.

4. The method of claim 1, wherein the dose is about 50 mg to about 100 mg.

5. The method of claim 1, wherein the dose is about 100 mg to about 200 mg.

6. The method of claim 1, wherein the dose is about 50 mg to about 200 mg.

7. The method of claim 1, wherein the compound is in a sodium salt form.

8. The method of claim 1, wherein the compound is administered by an injection.

9. The method of claim 8, wherein the injection is a subcutaneous injection.

10. The method of claim 1, wherein the compound is present in a single unit dosage form.

11. The method of claim 1, wherein the non-alcoholic fatty liver disease is non-alcoholic steatohepatitis (NASH).

12. The method of claim 1, wherein the human subject has a HSD17β13 rs72613567 mutation, PNPLA3 rs738409 (I148M) mutation, or a combination thereof.

13. The method of claim 1, wherein the double-stranded oligonucleotide comprises an antisense strand that comprises a modified nucleotide sequence that differs by 0 or 1 nucleotides from one of the following sequences (5′→3′):
(nucleotide sequence SEQ ID NO: 1)
usCfsas UfcUfaucagAfcUfuCfuUfaCfsg;
(nucleotide sequence SEQ ID NO: 5)
usCfsasUfcUfaUfcAfgAfcUfuCfuUfaCfsg;
(nucleotide sequence SEQ ID NO: 6)
usGfsasUfcCfaAfaAfaUfgUfcCfuAfgGfsc;
or
(nucleotide sequence SEQ ID NO: 7)
usGfsasUfcCfaaaaaUfgUfcCfuAfgGfsc;
and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

14. The method of claim 1, wherein the double-stranded oligonucleotide comprises a sense strand that comprises a modified nucleotide sequence that differs by 0 or 1 nucleotides from one of the following sequences (5′→3′):
(nucleotide sequence SEQ ID NO: 3)
cguaagaaGfuCfuGfauagauga;
(nucleotide sequence SEQ ID NO: 8)
cguaagaaGfUfCfugauagauga;
(nucleotide sequence SEQ ID NO: 9)
gccuaggaCfAfUfuuuugiauca;
or
(nucleotide sequence SEQ ID NO: 10)
gccuaggaCfaUfuUfuugiauca;
and wherein a, c, g, i, and u represent 2′-O-methyl adenosine, 2′-O-methyl cytidine, 2′-O-methyl guanosine, 2′-O-methyl inosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; and s represents a phosphorothioate linkage.

15. The method of claim 1, wherein the double-stranded oligonucleotide comprises an antisense strand that comprises a sequence (5′→3′): usCfsasUfcUfaucagAfcUfuCfuUfaCfsg (nucleotide sequence SEQ ID NO:1), and a sense strand that comprises a sequence (5′→3′):
(nucleotide sequence SEQ ID NO: 11)
(NAG37)s(invAb)scguaagaaGfuCfuGfauagaugas(invAb),
and wherein a, c, g, and u represent 2′-O-methyl adenosine, 2′-O-methyl cytidine, 2′-O-methyl guanosine, and 2′-O-methyl uridine, respectively; Af, Cf, Gf, and Uf represent 2′-fluoro adenosine, 2′-fluoro cytidine, 2′-fluoro guanosine, and 2′-fluoro uridine, respectively; s represents a phosphorothioate linkage; and (invAb) represents an inverted abasic deoxyribose residue.

16. The method of claim 1, wherein the method reduces the expression of HSD17β13 mRNA in the human subject by at least about 50% compared with that prior to the administration, as measured by quantitative Reverse Transcriptase Polymerase Chain Reaction (qRT-PCR) with a liver biopsy sample from the human subject.

17. The method of claim 1, wherein the method reduces the expression of HSD17β13 protein in the human subject by at least about 30% compared with that prior to the administration, as measured by Western Blot with a liver biopsy sample from the human subject.

18. The method of claim 1, wherein the method reduces ALT (alanine aminotransferase) level in the human subject by at least about 40% compared with that prior to the administration, as measured in the human subject's serum.

19. The method of claim 1, wherein the method reduces AST (aspartate aminotransferase) level in the human subject by at least about 20% compared with that prior to the administration, as measured in the human subject's serum.

20. The method of claim 1, wherein the method reduces or slows an increase of the human subject's liver fat fraction compared with that prior to the administration, as measured by magnetic resonance imaging.

21. The method of claim 1, wherein the method reduces or slows an increase of the human subject's liver stiffness compared with that prior to the administration, as measured by transient elastography.

22. The method of claim 1, wherein the chemical structure of the ligand comprises:

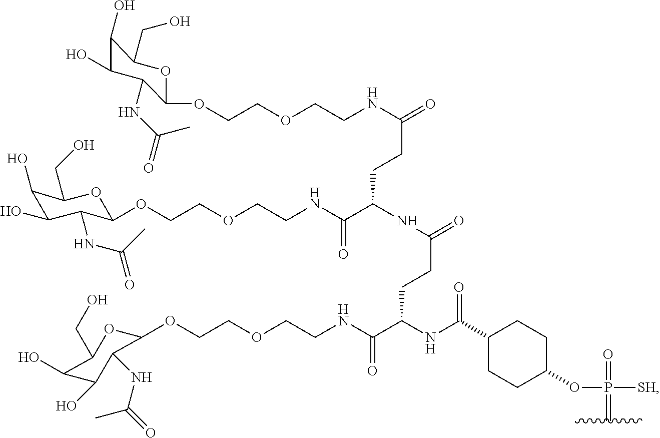

23. The method of claim 22, wherein the compound is in a free acid form having a chemical structure as illustrated in FIG. 5A to 5D.

24. The method of claim 1, wherein the chemical structure of the ligand comprises:

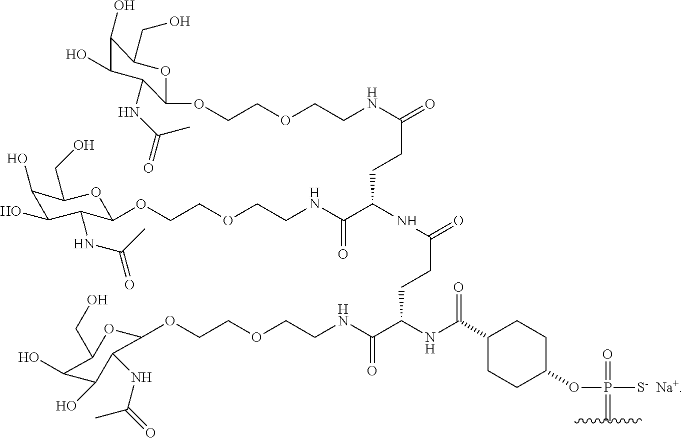

25. The method of claim 24, wherein the compound is in a sodium salt form having a chemical structure as illustrated in FIG. 6A to 6D.

26. The method of claim 1, wherein the dose is about 50 mg.

27. The method of claim 1, wherein the dose is about 100 mg.

28. The method of claim 1, wherein the dose is about 200 mg.

29. (canceled)

30. (canceled)

31. (canceled)

32. (canceled)


SEQUENCE LISTING (11 sequences)

SEQ ID NO: 1 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluorocytidine phosphorothioate
  modified_base 3: mod_base = OTHER; note = 2'-O-methyladenosine phosphorothioate
  modified_base 4: mod_base = OTHER; note = 2'-fluorouridine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methyladenosine
  modified_base 8: mod_base = OTHER; note = 2'-O-methyluridine
  modified_base 9: mod_base = OTHER; note = 2'-O-methylcytidine
  modified_base 10: mod_base = OTHER; note = 2'-O-methyladenosine
  modified_base 11: mod_base = OTHER; note = 2'-O-methylguanosine
  modified_base 12: mod_base = OTHER; note = 2'-fluoroadenosine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methyluridine
  modified_base 18: mod_base = OTHER; note = 2'-fluorouridine
  modified_base 19: mod_base = OTHER; note = 2'-O-methyladenosine
  modified_base 20: mod_base = OTHER; note = 2'-fluorocytidine phosphorothioate
  modified_base 21: mod_base = OTHER; note = 2'-O-methylguanosine
tcatctatcagacttcttacg

SEQ ID NO: 2 - RNA, 21 nt, synthetic construct
  source 1..21: mol_type = other RNA
tcatctatcagacttcttacg

SEQ ID NO: 3 - RNA, 21 nt, synthetic construct
  source 1..21: mol_type = other RNA
  modified_base 1: mod_base = OTHER; note = 2'-O-methylcytidine
  modified_base 2: mod_base = OTHER; note = 2'-O-methylguanosine
  modified_base 3: mod_base = OTHER; note = 2'-O-methyluridine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-O-methyluridine
  modified_base 11: mod_base = OTHER; note = 2'-fluorocytidine
  modified_base 12: mod_base = OTHER; note = 2'-O-methyluridine
  modified_base 13: mod_base = OTHER; note = 2'-fluoroguanosine
  modified_base 14: mod_base = OTHER; note = 2'-O-methyladenosine
  modified_base 15: mod_base = OTHER; note = 2'-O-methyluridine
  modified_base 16: mod_base = OTHER; note = 2'-O-methyladenosine
  modified_base 17: mod_base = OTHER; note = 2'-O-methylguanosine
  modified_base 18: mod_base = OTHER; note = 2'-O-methyladenosine
  modified_base 19: mod_base = OTHER; note = 2'-O-methyluridine
  modified_base 20: mod_base = OTHER; note = 2'-O-methylguanosine
  modified_base 21: mod_base = OTHER; note = 2'-O-methyladenosine
cgtaagaagtctgatagatga

SEQ ID NO: 4 - RNA, 21 nt, synthetic construct
  source 1..21: mol_type = other RNA
cgtaagaagtctgatagatga

SEQ ID NO: 5 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluorocytidine phosphorothioate
  modified_base 3: mod_base = OTHER; note = 2'-O-methyladenosine phosphorothioate
  modified_base 4: mod_base = OTHER; note = 2'-fluorouridine
  modified_base 5: mod_base = OTHER; note = 2'-O-methylcytidine
  modified_base 6: mod_base = OTHER; note = 2'-fluorouridine
  modified_base 7: mod_base = OTHER; note = 2'-O-methyladenosine
  modified_base 8: mod_base = OTHER; note = 2'-fluorouridine
  modified_base 9: mod_base = OTHER; note = 2'-O-methylcytidine
  modified_base 10: mod_base = OTHER; note = 2'-fluoroadenosine
  modified_base 11: mod_base = OTHER; note = 2'-O-methylguanosine
  modified_base 12: mod_base = OTHER; note = 2'-fluoroadenosine
  modified_base 13: mod_base = OTHER; note = 2'-O-methylcytidine
  modified_base 14: mod_base = OTHER; note = 2'-fluorouridine
  modified_base 15: mod_base = OTHER; note = 2'-O-methyluridine
  modified_base 16: mod_base = OTHER; note = 2'-fluorocytidine
  modified_base 17: mod_base = OTHER; note = 2'-O-methyluridine
  modified_base 18: mod_base = OTHER; note = 2'-fluorouridine
  modified_base 19: mod_base = OTHER; note = 2'-O-methyladenosine
  modified_base 20: mod_base = OTHER; note = 2'-fluorocytidine phosphorothioate
  modified_base 21: mod_base = OTHER; note = 2'-O-methylguanosine
tcatctatcagacttcttacg

SEQ ID NO: 6 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluoroguanosine phosphorothioate
  modified_base 3: mod_base = OTHER; note = 2'-O-methyladenosine phosphorothioate
  modified_base 4: mod_base = OTHER; note = 2'-fluorouridine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-fluoroadenosine
  modified_base 9: mod_base = OTHER; note = 2'-O-methyladenosine
  modified_base 10: mod_base = OTHER; note = 2'-fluoroadenosine
  modified_base 11: mod_base = OTHER; note = 2'-O-methyladenosine
  modified_base 12: mod_base = OTHER; note = 2'-fluorouridine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methyluridine
  modified_base 18: mod_base = OTHER; note = 2'-fluoroadenosine
  modified_base 19: mod_base = OTHER; note = 2'-O-methylguanosine
  modified_base 20: mod_base = OTHER; note = 2'-fluoroguanosine phosphorothioate
  modified_base 21: mod_base = OTHER; note = 2'-O-methylcytidine
tgatccaaaaatgtcctaggc

SEQ ID NO: 7 - RNA, 21 nt, synthetic construct
  source 1..21: mol_type = other RNA
  modified_base 1: mod_base = OTHER; note = 2'-O-methyluridine phosphorothioate
  modified_base 2: mod_base = OTHER; note = 2'-fluoroguanosine phosphorothioate
  modified_base 3: mod_base = OTHER; note = 2'-O-methyladenosine phosphorothioate
  modified_base 4: mod_base = OTHER; note = 2'-fluorouridine
  modified_base 5: mod_base = OTHER; note = 2'-O-methylcytidine
  modified_base 6: mod_base = OTHER; note = 2'-fluorocytidine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-O-methyladenosine
  modified_base 11: mod_base = OTHER; note = 2'-O-methyladenosine
  modified_base 12: mod_base = OTHER; note = 2'-fluorouridine
  modified_base 13: mod_base = OTHER; note = 2'-O-methylguanosine
  modified_base 14: mod_base = OTHER; note = 2'-fluorouridine
  modified_base 15: mod_base = OTHER; note = 2'-O-methylcytidine
  modified_base 16: mod_base = OTHER; note = 2'-fluorocytidine
  modified_base 17: mod_base = OTHER; note = 2'-O-methyluridine
  modified_base 18: mod_base = OTHER; note = 2'-fluoroadenosine
  modified_base 19: mod_base = OTHER; note = 2'-O-methylguanosine
  modified_base 20: mod_base = OTHER; note = 2'-fluoroguanosine phosphorothioate
  modified_base 21: mod_base = OTHER; note = 2'-O-methylcytidine
tgatccaaaaatgtcctaggc

SEQ ID NO: 8 - RNA, 21 nt, synthetic construct
  source 1..21: mol_type = other RNA
  modified_base 1: mod_base = OTHER; note = 2'-O-methylcytidine
  modified_base 2: mod_base = OTHER; note = 2'-O-methylguanosine
  modified_base 3: mod_base = OTHER; note = 2'-O-methyluridine
  modified_base 4: mod_base = OTHER; note = 2'-O-methyladenosine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluoroguanosine
  modified_base 10: mod_base = OTHER; note = 2'-fluorouridine
  modified_base 11: mod_base = OTHER; note = 2'-fluorocytidine
  modified_base 12: mod_base = OTHER; note = 2'-O-methyluridine
  modified_base 13: mod_base = OTHER; note = 2'-O-methylguanosine
  modified_base 14: mod_base = OTHER; note = 2'-O-methyladenosine
  modified_base 15: mod_base = OTHER; note = 2'-O-methyluridine
  modified_base 16: mod_base = OTHER; note = 2'-O-methyladenosine
  modified_base 17: mod_base = OTHER; note = 2'-O-methylguanosine
  modified_base 18: mod_base = OTHER; note = 2'-O-methyladenosine
  modified_base 19: mod_base = OTHER; note = 2'-O-methyluridine
  modified_base 20: mod_base = OTHER; note = 2'-O-methylguanosine
  modified_base 21: mod_base = OTHER; note = 2'-O-methyladenosine
cgtaagaagtctgatagatga

SEQ ID NO: 9 - RNA, 21 nt, synthetic construct
  source 1..21: mol_type = other RNA
  modified_base 1: mod_base = OTHER; note = 2'-O-methylguanosine
  modified_base 2: mod_base = OTHER; note = 2'-O-methylcytidine
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-fluoroadenosine
  modified_base 11: mod_base = OTHER; note = 2'-fluorouridine
  modified_base 12: mod_base = OTHER; note = 2'-O-methyluridine
  modified_base 13: mod_base = OTHER; note = 2'-O-methyluridine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methylguanosine
  modified_base 17: mod_base = OTHER; note = 2'-O-methylinosine
  modified_base 18: mod_base = OTHER; note = 2'-O-methyladenosine
  modified_base 19: mod_base = OTHER; note = 2'-O-methyluridine
  modified_base 20: mod_base = OTHER; note = 2'-O-methylcytidine
  modified_base 21: mod_base = OTHER; note = 2'-O-methyladenosine
gcctaggacatttttgnatca

SEQ ID NO: 10 - RNA, 21 nt, synthetic construct
  source 1..21: mol_type = other RNA
  modified_base 1: mod_base = OTHER; note = 2'-O-methylguanosine
  modified_base 2: mod_base = OTHER; note = 2'-O-methylcytidine
  modified_base 3: mod_base = OTHER; note = 2'-O-methylcytidine
  modified_base 4: mod_base = OTHER; note = 2'-O-methyluridine
  modified_base 5: mod_base = OTHER; note = 2'-O-methyladenosine
  modified_base 6: mod_base = OTHER; note = 2'-O-methylguanosine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-fluorocytidine
  modified_base 10: mod_base = OTHER; note = 2'-O-methyladenosine
  modified_base 11: mod_base = OTHER; note = 2'-fluorouridine
  modified_base 12: mod_base = OTHER; note = 2'-O-methyluridine
  modified_base 13: mod_base = OTHER; note = 2'-fluorouridine
  modified_base 14: mod_base = OTHER; note = 2'-O-methyluridine
  modified_base 15: mod_base = OTHER; note = 2'-O-methyluridine
  modified_base 16: mod_base = OTHER; note = 2'-O-methylguanosine
  modified_base 17: mod_base = OTHER; note = 2'-O-methylinosine
  modified_base 18: mod_base = OTHER; note = 2'-O-methyladenosine
  modified_base 19: mod_base = OTHER; note = 2'-O-methyluridine
  modified_base 20: mod_base = OTHER; note = 2'-O-methylcytidine
  modified_base 21: mod_base = OTHER; note = 2'-O-methyladenosine
gcctaggacatttttgnatca

SEQ ID NO: 11 - RNA, 23 nt, synthetic construct
  source 1..23: mol_type = other RNA
  modified_base 1: mod_base = OTHER; note = tridentate N-acetyl-galactosamine-containing targeting ligand modified inverted abasic nucleotide phosphorothioate
  modified_base 2: mod_base = OTHER; note = 2'-O-methylcytidine
  modified_base 3: mod_base = OTHER; note = 2'-O-methylguanosine
  modified_base 4: mod_base = OTHER; note = 2'-O-methyluridine
  modified_base 5: mod_base = OTHER; note = 2'-O-methyladenosine
  modified_base 6: mod_base = OTHER; note = 2'-O-methyladenosine
  modified_base 7: mod_base = OTHER; note = 2'-O-methylguanosine
  modified_base 8: mod_base = OTHER; note = 2'-O-methyladenosine
  modified_base 9: mod_base = OTHER; note = 2'-O-methyladenosine
  modified_base 10: mod_base = OTHER; note = 2'-fluoroguanosine
  modified_base 11: mod_base = OTHER; note = 2'-O-methyluridine
  modified_base 12: mod_base = OTHER; note = 2'-fluorocytidine
  modified_base 13: mod_base = OTHER; note = 2'-O-methyluridine
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-O-methyluridine
  modified_base 17: mod_base = OTHER; note = 2'-O-methyladenosine
  modified_base 18: mod_base = OTHER; note = 2'-O-methylguanosine
  modified_base 19: mod_base = OTHER; note = 2'-O-methyladenosine
  modified_base 20: mod_base = OTHER; note = 2'-O-methyluridine
  modified_base 21: mod_base = OTHER; note = 2'-O-methylguanosine
  modified_base 22: mod_base = OTHER; note = 2'-O-methyladenosine phosphorothioate
  modified_base 23: mod_base = OTHER; note = Inverted abasic nucleotide
ncgtaagaagtctgatagatgan